(19) United States
(12) Patent Application Publication
(10) Pub. No.: US 2022/0204970 A1
(43) Pub. Date: Jun. 30, 2022

(54) POLYVALENT GUIDE RNAS FOR CRISPR ANTIVIRALS

(71) Applicant: The University of North Carolina at Greensboro, Greensboro, NC (US)

(72) Inventor: Eric JOSEPHS, Durham, NC (US)

(21) Appl. No.: 17/556,481

(22) Filed: Dec. 20, 2021

Related U.S. Application Data
(60) Provisional application No. 63/128,453, filed on Dec. 21, 2020.

(51) Int. Cl. C12N 15/11; C12N 15/10; C12N 9/22; A61P 31/14

(52) U.S. Cl. CPC C12N 15/111; C12N 15/102; C12N 2310/20; A61P 31/14; C12N 9/22

(57) ABSTRACT

Generally, the present disclosure is directed to methods for gRNA design and products thereof that can be used as antivirals in which the produced gRNAs can be tolerant to polymorphisms across clinical strains and/or adapted for activity at multiple viral sites. Aspects of example gRNAs can also include reduced interactions with the human genome or transcriptome.

### CROSS REFERENCE TO RELATED APPLICATIONS

The present application claims priority to U.S. Provisional Application Ser. No. 63/128,453, filed Dec. 21, 2020, the contents and substance of which are incorporated herein in their entirety by reference.

### STATEMENT REGARDING FEDERALLY SPONSORED RESEARCH

This invention was made with government support under contract nos. A20-0074-001 and RAMSeS 19-0113 awarded by the National Institute of Health and National institute of General Medical Sciences, respectively. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 12, 2022, is named UNCG_20-0009_SL.txt and is 19,426 bytes in size.

### FIELD

The present disclosure relates to methods for designing gRNAs for use in applications such as antivirals.

### BACKGROUND

Class II CRISPR effectors like Cas9, Cas12, and Cas13, are endonucleases that use a modular segment of their RNA cofactors known as CRISPR RNAs (crRNAs) or guide RNAs (gRNAs) to recognize and trigger the degradation of nucleic acids with a sequence complementary to that segment. These diverse enzymes are derived from a bacterial and archaeal defensive response to invasive plasmids and viruses and, because of their ability to be easily redirected to nucleic acid with different sequences by simply changing the sequence composition of that short portion of their gRNAs called their ‘spacer,’ they have been re-appropriated over the past several years for a number of different biotechnological applications, most notably in precision gene editing. During precision gene editing, a CRISPR effector is transfected into a human cell and directed to introduce a double strand break (DSB) into the genomic DNA at a specific targeted sequence; genomic mutations have been introduced at those sites as a result of mutagenic DSB repair. These technologies have experienced widespread adoption for biomedical research and possess a number of emerging therapeutic applications as well.

Another nascent, but less-developed, application of CRISPR effectors has been as novel antiviral therapeutics, diagnostics, and prophylactics, based on their ability to recognize and degrade viral genomes. The first CRISPR antiviral efforts used the type II CRISPR effector Cas9 from Streptococcus pyogenes (SpyCas9), which recognizes and introduces DSBs into double-stranded DNA (dsDNA) targets, and so efforts were focused largely on degrading dsDNA viruses and excising the Human immunodeficiency virus 1 (HIV-1) proviruses from cells with latent infection. However, it was found that rapid accumulation of mutations within the target regions inhibit CRISPR activity and can drive mutagenic escape from these treatments, and so successful application of these efforts has been limited. Later, another variety of CRISPR effectors, type V CRISPR effector Cas12a (formerly named Cpf1), was identified as a divergent class of RNA-guided dsDNA endonucleases that are also capable of precision gene editing activities. Recently, it was reported that Cas12a effectors can outperform Cas9 in HIV inhibition studies in vitro. Cas12a effectors were also found to indiscriminately degrade single-stranded DNA (ssDNA) after recognizing its dsDNA target, and several sensitive viral detection technologies have been developed that make use of this capability. Furthermore, because the vast majority of pathogenic viruses are RNA viruses, more recently excitement for the potential of CRISPR antivirals has been spurred by the development of RNA-guided RNA endonucleases, in particular type VI CRISPR effectors known as Cas13a (formerly C2c2), Cas13b, and Cas13d, for applications in human cells. Recent demonstrations of Cas13 reducing viral load by either degrading viral single-stranded RNA (ssRNA) genomes or viral mRNA have been performed in plant (e.g., turnip mosaic virus), mammalian (e.g., dengue virus), porcine reproductive and respiratory syndrome virus, and human cells (e.g., lymphocytic choriomeningitis virus); influenza A virus; and vesicular stomatitis virus, and severe acute respiratory syndrome coronavirus 2 (SARS-CoV-2). Cas13 nucleases also exhibit nonspecific RNAse activity after recognition of their targets, and this nonspecific degradation has been exploited in sensitive viral detection strategies as well. These applications have shown significant promise for the future of CRISPR antivirals; however, further maturation of these biotechnologies is required to overcome some of the remaining challenges to reach their full potential.

One major challenge in the development of CRISPR antivirals comes from the rapid mutation rate of viruses. As a result, CRISPR antivirals must be tolerant to polymorphisms that occur across viral strains, and CRISPR antiviral systems also must be designed to suppress mutational escape. Previously, these challenges have been addressed by targeting the CRISPR effector to highly conserved regions of the viral genome, and by the introduction of multiple gRNAs to target different regions of the viral genome simultaneously (multiplexing) in order to make mutational escape less likely. At the same time, CRISPR multiplexing introduces a number of additional practical challenges. Furthermore, no quantitative criteria have been described for the level of sequence conservation, beyond counting the number of inter-strain variations at different genomic locations, for identifying potential antiviral targets expected to be highly active across clinical variants.

### SUMMARY

The present disclosure is directed to methods of gRNA design and nucleic acid sequences derived therefrom. In particular, the present disclosure provides methods for designing the sequences of polyvalent guide RNAs (pgRNAs).

An example aspect of the present disclosure can include a method to improve the breadth, range, and efficiency of CRISPR antivirals and CRISPR-based virus detection by improving the design and selection of the guide RNA. The disclosure is based on the idea that CRISPR effectors are inherently “promiscuous” (able to degrade non-perfect complements, subject to a number of biophysical constraints) as a result of their origins in bacterial defense against phages, and this promiscuity can be exploited in the design of gRNAs that might more effectively be able to target a broad range of coronaviruses (or viral families more broadly) or even multiple sites within the same viral genome in order to potentially enhance anti-viral activity.

The off-target activities of CRISPR systems have been noted in gene editing technologies, where off-target activity can a major hindrance to therapeutic applications, however there have been few applications of this knowledge. Example embodiments herein can be applied for identifying widely conserved ‘targets’, which are sequences (partially) complementary to the gRNA but which may have mutations in some strains at parts of the target where mutations are well tolerated, as one of the primary design considerations of a gRNA, rather than locations of conserved sequence (where mutations might not at all affect CRISPR activity).

Further, one example aspect of the present disclosure includes methods to balance the promiscuity of guide RNA to reduce possible promiscuous activity with the human genome (DNA) or transcriptome (RNA). In some implementations, these considerations can also be balanced against other biophysical factors that might affect CRISPR activity, such as any predicted secondary structures of the guide RNA, polynucleotide repeats that might affect expression or structure, accessibility of the targeted sites, activity prediction from other sources.

While, CRISPR antivirals have not been validated for therapeutic application, there are a number of in vitro reports. The therapeutic potential of CRISPR antivirals is emerging and there will likely be increased interest in the wake of the COVID-19 pandemic. Such antivirals may be of particular interest in cases of emerging pathogenic viruses, like SARS-COV-2, where no vaccine exists and limited treatments exist. CRISPR antivirals could provide a very rapid response therapeutic under these conditions.

The same CRISPR effectors (e.g., Cas 13) that have been used for in vitro antivirals have also been used for the rapid detection of pathogenic viruses from human samples, so another example aspect of the present disclosure can include detection systems for targeting a virus.

In general, the present disclosure is directed to various embodiments which can include, for example, a method for determining a pgRNA sequence. For instance, an example method can include identifying two or more target sequences (nucleic acid sequence can be RNA and/or DNA) in a viral genome for recognition by a Cas effector, and for each target sequence of the two or more target sequences, calculating a homology score comprising aligning said target sequence with each other target sequence of the two or more target sequences. After calculating the homology score, the example method can also include determining one or more target pairs based at least in part on the homology score, where each target pair includes a first target sequence and a second target sequence of the two or more target sequences having the homology score calculated as greater than or equal to 60% sequence identity (e.g., greater than or equal to 75, 80, 85, or 95% sequence identity). Additionally, the example method can include generating a pgRNA template for at least one of the one or more target pairs, where the pgRNA template has a complementary sequence to the first target sequence, a complementary sequence the second target sequence, or a convergent sequence (e.g., a sequence that is some combination of both complementary sequences). Another aspect of the example method can include generating a relative activity score for each of one or more pgRNA templates by comparing the pgRNA template to a complementary sequence to the first target sequence and a complementary sequence to a second nucleotide sequence present in a different viral genome, a mutant viral genome, or both, wherein each pgRNA template comprises a sequence of nucleotides. The example method can optionally include determining an off-target score for each pgRNA template based at least in part on the relative activity score generated for said pgRNA template. Finally, the example method can include determining the pgRNA sequence based at least in part on the relative activity score for each pgRNA template, the off-target score, or both.

An example aspect of identifying the two or more target sequences in the viral genome can include determining a sequence position for each of one or more protospacer motifs present in the viral genome based at least in part on the CAS effector, were each of the one or more protospacer motifs include an adjacent sequence of nucleotides; and assigning at least one sequence position as a protospacer position; and identifying the two or more target sequences as a sequence of nucleotides immediately downstream (toward the 3′ end) of the protospacer position.

For certain example methods the Cas effector can be enAsCas12a.

In some example methods the one or more protospacer motifs are from the group consisting of: TTYN, CTTV, RTTC, TATM, CTCC, TCCC, TACA, RTTS, TATA, TGTV, ANCC, CVCC, TGCC, GTCC, TTAC, or combinations thereof.

In some example methods, the one or more protospacer motifs are from the group consisting of: TTYN, CTTV, RTTC, TATM, CTCC, TCCC, TACA, or combinations thereof.

In some example methods, the different viral genome and the viral genome are included in a viral family (e.g., coronaviruses).

An example aspect comparing the pgRNA template to the complementary sequence to the first nucleotide sequence and the complementary sequence to the second nucleotide sequence present in the different viral genome, the mutant viral genome, or both can include determining a first sequence identify for the pgRNA template to the complementary sequence to the first nucleotide sequence and a second sequence identity for the pgRNA template to the complementary sequence to the second nucleotide sequence. In certain example methods the first sequence identity and the second sequence identity are calculated based on a BLAST alignment, and wherein the relative activity score is based at least in part on the first sequence identity and the second sequence identity.

In some example methods calculating the off-target score is performed only for the pgRNA templates having calculated the first sequence identity as greater than about 60% and the second sequence identity as greater than about 60%. For instance, in certain example methods, calculating the off-target score is performed only for the pgRNA templates having calculated the first sequence identity as greater than about 90% and the second sequence identity as greater than about 90%.

For certain example methods, calculating the off-target score is based at least in part on comparing each of the one or more pgRNA templates to a human genome sequence or a human transcriptome sequence.

For certain example methods, determining the pgRNA sequence is based at least in part on a region of interest comprising a sequence of adjacent nucleotides present in the viral genome.

Another example embodiment of the present disclosure can include a pgRNA sequence determined according to any of the preceding example methods. For instance, a pgRNA can be determined based on identifying two or more target sequences in a coronavirus genome (e.g., SARS-CoV-2).

A further example embodiment of the present disclosure can include a method for treating a viral infection in a patient that includes delivering to a patient in need thereof a composition including an example pgRNA having a sequence determined according to example methods herein.

Aspects of certain methods for treating a viral infection can include treating a patient displaying certain symptoms (e.g., Covid-19).

### BRIEF DESCRIPTION OF THE DRAWINGS

FIG. 1A illustrates a cartoon of gRNA design for targeted gene editing in accordance with prior aspects.

FIG. 1B illustrates a cartoon of pgRNA design in accordance with aspects of the present disclosure.

FIG. 1C illustrates a cartoon displaying an example aspect of pgRNA design in accordance with embodiments of the present disclosure. FIG. 1C discloses SEQ ID NOS 39-40, respectively, in order of appearance.

FIG. 2 illustrates a flow chart diagram of an example method for designing pgRNA in accordance with example embodiments of the present disclosure. FIG. 2 discloses SEQ ID NOS 8-9, respectively, in order of appearance.

FIG. 3A illustrates a graph an example for determining target pairs for a Cas effector in accordance with example aspects of the present disclosure.

FIG. 3B illustrates a graph displaying sequence conservation (SC) across viral genomes in accordance with example aspects of the present disclosure.

FIG. 3C illustrates a graph displaying minimal relative activity across variants (MRAV) predicted for a Cas effector in accordance with example aspects of the present disclosure.

FIG. 4 illustrates a bar graph displaying estimated relative CRISPR activity in accordance with example aspects of the present disclosure.

FIG. 5A illustrates a graph displaying an example for determining target pairs for a Cas effector in accordance with example aspects of the present disclosure.

FIG. 5B illustrates a bar graph displaying estimated relative CRISPR activity in accordance with example aspects of the present disclosure.

FIG. 6 illustrates a stained gel displaying example in vitro validation data in accordance with example aspects of the present disclosure. FIG. 6 discloses SEQ ID NOS 22 and 20, respectively, in order of appearance.

FIG. 7A illustrates a cartoon showing design of pgRNAs for targeting pairs of sequences.

FIG. 7B illustrates pairs of targets in the TRBO-GFP for the different pgRNAs. FIG. 7B discloses SEQ ID NOS 41-47, respectively, in order of appearance.

FIG. 7C illustrates images of leaves of N. bethamiana were infiltrated with a composition including plasmids for producing gRNA.

FIGS. 7D-7E illustrate graphs displaying data for relative viral GFP RNA level. FIG. 7D discloses SEQ ID NOS 50 and 49, respectively, in order of appearance.

FIG. 8A illustrates a representation of Cas binding and activity.

FIG. 8B illustrates a table and data representing detectable collateral activity. FIG. 8B discloses SEQ ID NOS 51-65, respectively, in order of appearance.

FIG. 8C illustrates example pgRNAs designed to target (+) ssRNA virus SARS-CoV-2. FIG. 8C discloses SEQ ID NOS 66-71, respectively, in order of appearance.

FIG. 8D illustrates a graph displaying fluorescence data.

FIG. 8E illustrates graphs displaying data from a SHERLOCK-type Cas13 viral diagnostic assay.

FIG. 8F illustrates a representation showing Cas9 recognizes and cleaves dsDNA.

FIG. 8G illustrates a pgRNA designed to target two sequences derived from the Tobacco Rattle Virus.

FIG. 8H illustrates a sequence comparison showing divergence of targets A and B compared to a pgRNA. FIG. 8H discloses SEQ ID NOS 22, 21 and 20, respectively, in order of appearance.

FIG. 8I illustrates example data from a gel assay.

FIG. 8J illustrates pgRNA sequence and percent cleaved by Cas9 data. FIG. 8J discloses SEQ ID NOS 72-75, 73, 76-77, 73, 78-79, 73, 80-95, 48-49 and 1, respectively, in order of appearance.

### DETAILED DESCRIPTION

In general, the present disclosure is directed to methods for design of gRNAs for CRISPR antivirals that exploits the widely-recognized tendency of different CRISPR effectors to possess varying levels tolerances to imperfect complementary between the gRNA spacer and the targets. While significant efforts have gone into limiting this tendency for precision gene editing applications—and activity at multiple or “off-target” sites prevented at all costs—implementations of the present disclosure utilize a process for generating “polyvalent” gRNA (pgRNAs) that can demonstrate activity at multiple viral genomic sites: in effect producing operational multiplexing with a single gRNA. For instance, embodiments of the present disclosure can be used to generate pgRNA sequences that can be characterized by one or more of the following properties: (i) high relative activity at multiple viral targets, (ii) high relative activity across clinical strain variants, (iii) low predicted relative activity at potential human “off-targets,” and (iv) reasonable biophysical characteristics that suggest high CRISPR activity for potential antiviral and/or viral detection applications.

Aspects of example implementations include: designing pgRNAs which exhibit >95% activity at distant viral sites along a viral genome such as the SARS-CoV-2 ssRNA genome and which can be tolerant to variations across strains, while still avoiding predicted off-target activity with components of the human transcriptome. In particular, these pgRNAs may be designed based on the pgRNA use in combination with a specific Cas effector such as Cas13 from Ruminococcus flavefaciens XPD3002 (RfxCas13d). Another example of a Cas effector can include a Cas12a variant (engineered Cas12a from Acidaminococcus sp. BV3L6, enAsCas12a) that can target multiple locations along the HIV-1 provirus—up to three viral targets using a single pgRNA designed in accordance with the present disclosure—while minimizing activity at other sites in the human genome.

One example implementation in accordance with the present disclosure can include a method for determining a pgRNA sequence, such as a pgRNA sequence for producing an antiviral. The method for determining a pgRNA sequence can include identifying two or more target sequences (e.g., a nucleic acid sequence that can be RNA or DNA) in a viral genome for recognition by a CAS effector. The method can also include calculating a homology score, based on performing an alignment between each target sequence of the two or more target sequences with each other target sequence. More particularly, the homology score can include a metric such as sequence identify, sequence similarity, or other similar method for determining regions of overlap between target sequences.

Example methods for determining a pgRNA sequence can also include determining a target pair comprising a first nucleotide sequence present in the viral genome and a second nucleotide sequence present in the different viral genome, the mutant viral genome, or both. In some embodiments, the target pair can be determined based at least in part on the homology score. For example, the homology score may determine that a sequence of nucleotides (nt) displays 95% sequence identity between the viral genome and a different viral genome. In certain implementations, depending on if the homology score meets a certain threshold (e.g., greater than 90%, 91%, 92%, 93%, 94%, 95%, 96%, 97%, 98%, or 99%), the sequence of nucleotides can be used to determine the target pair. As should be understood, the different viral genome may include a viral genome from the same viral family (e.g., coronaviruses).

Another aspect of example methods for determining a pgRNA sequence can include generating a relative activity score for each of one or more pgRNA templates by comparing the pgRNA template to a complementary sequence to the first nucleotide sequence and a complementary sequence to the second nucleotide sequence. The pgRNA templates can be generated by various means including random generation, computer modeling, or both, and generally each pgRNA template includes a sequence of nucleotides.

Example methods for determining a pgRNA sequence may further include determining whether to calculate an off-target score for each pgRNA template based at least in part on the relative activity score generated for said pgRNA template.

For example embodiments according to the present disclosure, determining the pgRNA sequence can based at least in part on the relative activity score for each pgRNA template, the off-target score, or both.

One example aspect of identifying the two or more target sequences in the viral genome can include determining a sequence position for each of one or more protospacer motifs present in the viral genome based at least in part on the Cas effector. For instance, certain Cas effectors may display preferential recognition and/or binding to different regions of the viral genome (e.g., protospacer motifs). In particular, some implementations may use the position of protospacer motifs in the viral genome to identify possible target sequences that would display improved efficacy for antiviral treatments. For example, by assigning at least one sequence position as a protospacer position, certain embodiments may identify the two or more target sequences as at least including a sequence of nucleotides immediately downstream of the protospacer position in the viral genome.

For implementations of the present disclosure, the Cas effector can include any Cas effector that can be implemented as part of a CRISPR system to result in breakage of nucleotide oligomers such as RNA or DNA. Some non-limiting examples of Cas effectors that can be used in embodiments of the disclosure include enAsCas12a (Cas12a), RfxCas13d (Cas 13d), and/or SpyCas9 (Cas 9).

As previously discussed, certain Cas effectors may display preferred recognition and/or binding to certain protospacer motifs. For instance, using a Cas effector of the present disclosure, the one or more protospacer motifs can include one or more from the group: TTYN, CTTV, RTTC, TATM, CTCC, TCCC, TACA, RTTS, TATA, TGTV, ANCC, CVCC, TGCC, GTCC, TTAC, or combinations thereof. In some implementations, the one or more protospacer motifs can include a subset of this group. For example, in certain embodiments, the one or more protospacer motifs are from the group: TTYN, CTTV, RTTC, TATM, CTCC, TCCC, TACA, or combinations thereof. More particularly, some embodiments can include identifying target sequences that occur downstream of the position of one or more of these protospacer motifs in the viral genome. As used herein, protospacer motifs are provided as nucleotide sequences: A—adenosine, C—cytosine, T—thymidine, G—guanosine, V—uridine, N—any nucleotide, R—adenosine or guanosine, S—guanosine or cytosine, Y—a pyrimidine (C, T, or V).

One aspect of example embodiments can include methods for developing pgRNA that can target members of a viral family. For instance, in some implementations, the viral genome and the different viral genome can be included in the same viral family. Viral families are similar to animal families in that the genomes of viruses of the same family display some degree of overlap which can be determined based on aligning the genetic sequence to determine the sequence identity or similarity for regions of the genome. One non-limiting example of a viral family can include coronaviruses (coronaviridae), which includes members such as SARS-CoV-2, MERS-CoV, and SARS-CoV. Another non-limiting example of a viral family can include retroviruses (retroviridae), which includes members such as human immunodeficiency virus (HIV) and human T-lymphotropic virus (HTLV).

In certain implementations, methods for determining a pgRNA sequence can include identifying target sequences in a viral genome from a certain viral family and, calculating a homology score between a first viral genome from the certain viral family and a second, different viral genome from the same certain viral family. As an example for illustration, the first viral genome can be the genome for SARS-CoV-2 and the second viral genome can be the genome for MERS-CoV.

According to an aspect of certain embodiments, comparing the pgRNA template to a complementary sequence to the first nucleotide sequence and a complementary sequence to the second nucleotide sequence can include determining a first sequence identify for the pgRNA template to the complementary sequence to the first nucleotide sequence and a second sequence identity for the pgRNA template to the complementary sequence to the second nucleotide sequence. In general, a complementary sequence as used herein carries the ordinary meaning in biology. Base paring rules for nucleotides indicate that each one of the 5 nucleobases (adenosine ‘A’, guanosine ‘G’, cytidine ‘C’, uridine ‘U’, thymidine ‘T’) has a complementary nucleobase based on the type of nitrogenous base. For example, the complement to A is T or U (and vice-versa) and the complement to C is G (and vise-versa). Thus a complementary sequence to the example oligonucleotide AUCGCAUCU can be XAGCGXAGA where ‘X’ is independently T or U. In determining whether the complement to A is T or U, the type of viral genetic material may be used as one basis. In certain embodiments for designing pgRNA, the complement to A may only be U.

For some example embodiments of the present disclosure, the first sequence identity and/or the second sequence identity can be determined according to various methods. One example method can include performing a sequence alignment such as a BLAST alignment. BLAST alignment is a tool for comparing two sequences (e.g., nucleotide sequences) to determine characteristics such as sequence identity or sequence similarity as measures of overlap between portions of the sequences. In this manner, regions of higher overlap (greater similarity) and regions of poor overlap (lower similarity) can be determined. Thus these regions of greater similarity may be used to design pgRNA that can target multiple viruses. As such, in some embodiments of the present disclosure, the relative activity score can be based at least in part on the first sequence identity and the second sequence identity.

In certain example embodiments, calculating the off-target score can be performed only for pgRNA templates having calculated the first sequence identity as greater than about 60% and the second sequence identity as greater than about 60%, such as the first sequence identity greater than 62%, 64%, 66%, 68%, 70%, 72%, 74%, 76%, 78%, 80%, 82%, 84%, 86%, 88%, 90%, 92%, 94%, 96%, or 98% and, independently, the second sequence identity greater than 62%, 64%, 66%, 68%, 70%, 72%, 74%, 76%, 78%, 80%, 82%, 84%, 86%, 88%, 90%, 92%, 94%, 96%, or 98%. For instance, in some implementations, calculating the off-target score is performed only for the pgRNA templates having calculated the first sequence identity as greater than about 90% and the second sequence identity as greater than about 90%.

An aspect of some implementations may include calculating the off-target score based at least in part on comparing each of the one or more pgRNA templates to a human genome sequence or a human transcriptome sequence. Generally, the off-target score can be used to approximate overlapping or possible reactivity between the designed pgRNA and genetic material (e.g., RNA or DNA) present in humans. In this manner, overlapping reactivity may be diminished by excluding or removing pgRNA templates meeting an off-target score threshold.

Another aspect of certain implementations can include using further selection criteria in the design of pgRNAs. For instance, determining the pgRNA sequence can based at least in part on a region of interest which includes a sequence of adjacent nucleotides present in the viral genome. The region of interest can include a position of a gene that may be of clinical or functional significance, a position which is conserved over many viral strains and or that demonstrates greater intolerance to mutations, or a position determined using an activity prediction such as one that can be performed using bioinformatic tools and/or methods, prior to experimental validation.

While the present application is generally directed to embodiments for treating humans, it should be understood that similar protocols may be developed for treating viral diseases in a variety of organisms. For example, viral prophylaxis and/or treatment is particularly needed in many agriculturally important plants and animals. One aspect of implementations for designing pgRNA for these organisms is modifying the step for calculating the off-target score. For the organism to be treated, the off-target score should be based on the alignment to the genome or transcriptome of the host organism to be treated (e.g., a plant genome). In this manner, implementations of the present disclosure can include pgRNA designed according to such example method that can be delivered to a plant to treat a viral infestation. Further, genetic modification of organisms including plants, may be used to create transgenic organisms that produce the pgRNA rather than requiring a delivery method.

One example embodiment of the present disclosure can include a pgRNA having a pgRNA sequence determined according to example embodiments of the present disclosure. Aspects of the pgRNA can include improved activity across multiple viral strains (e.g., viruses from the same viral family). For instance, the pgRNA can be included as a cofactor in a CRISPR-Cas system to produce an antiviral.

Aspects of the pgRNA can include a pgRNA sequence that is determined based on identifying two or more target sequences in a coronavirus genome (e.g., SARS-CoV-2).

Another example embodiment of the present disclosure can include a method for treating a viral infection by delivering to a patient in need thereof a composition comprising a pgRNA, the pgRNA having a pgRNA sequence determining according to example methods of the present disclosure. For instance, an example implementation of the present disclosure can include a method for treating a patient displaying symptoms of Covid-19, by delivering a composition including a pgRNA sequence determined based on identifying one or more sequences in the SARS-CoV-2 genome.

As described in the disclosure, sequence identity is related to sequence homology. Homology comparisons may be conducted by eye, or more usually, with the aid of readily available sequence comparison programs. These commercially and publicly available computer programs can be used to determine percent (%) homology between two or more sequences and may also calculate the sequence identity shared by two or more nucleic acid sequences. Sequence homologies may be generated by any of a number of computer programs known in the art, for example BLAST. BLAST and are available for offline and online searching (see e.g., https://blast.ncbi.nlm.nih.gov/Blast.cgi). As used herein, sequence identity values

further embodiment of the present disclosure can include a diagnostic that includes one or more pgRNA sequences designed according to example implementations of the present disclosure. These diagnostics can include viral detection platforms which can provide advantages such as more sensitive identification of viral genetic material (e.g., by increasing the effective numbers of viral targets in a clinical sample), improved time-to-detection, and diagnostics that are more robust to viral mutations and variations across viral strains. When these example CRISPR diagnostic effectors recognize a viral nucleic acid sequence complementary to their gRNA, they cleave the viral nucleic acids, then begin to indiscriminately degrade any other single-stranded RNA or DNA they encounter. In a CRISPR-based viral detection platform, a “probe” nucleic acid is attached to a molecule that becomes highly fluorescent when the probes are degraded indiscriminately by the CRISPR effector. When these probes are included and this reaction is coupled with an isothermal PCR reaction to increase the amount of viral nucleic acids present in a clinical sample, it rapidly produces a bright signal without the need for a thermocycler.

The present invention will be better understood with reference to the following non-limiting examples.

### EXAMPLES

The present examples provide aspects of embodiments of the present disclosure. These examples are not meant to limit embodiments solely to such examples herein, but rather to illustrate some possible implementations.

### Material and Methods

### Viral Nucleotide Sequences

The Severe acute respiratory syndrome coronavirus 2 (SARS-CoV-2) isolate Wuhan-Hu-1 complete genome (NCBI Reference Sequence: NC_045512.2) served as the primary target for pgRNA development vs. the SARS-CoV-2 ssRNA genome. Design of pgRNA targets vs. HIV-1 provirus used the Human immunodeficiency virus type 1 (HXB2), complete genome; HIV1/HTLV-III/LAV reference genome (GenBank: K03455.1).

### Calculation of Mismatch Penalties and Relative CRISPR Activities

Estimates of the relative CRISPR activity at sites not perfectly targeted by the gRNA/pgRNA spacer sequence were generated by calculating the Cutting Frequency Determination (CFD) score (35,45). To calculate the CFD score, the penalty (relative reduction in CRISPR activities) that result from each site with a mismatch is first drawn from a CFD matrix, the table of position-specific reductions of activity that occur as a result of mispairing between specific nucleotides in the spacer and target. The CFD matrices for CRISPR effector were generated by the Sanjana lab (RfxCas13d) and Doench lab (SpyCas9 and enAsCas12a, using the data from the “dropout” experiments) using massively parallel screens of gRNA libraries for CRISPR activity, and CFD scoring implemented in MATLAB using publicly available data sets from those labs. The CFD score for a given target and gRNA spacer is the product of the CFD penalties for each mismatch; the position-specific penalties (average over all possible mismatched nucleotides). This approach is fast to implement and has been successfully used as a reasonable approximation for CRISPR activity at off-target sites by for a number of different CRISPR effectors. The effect of different PAMs (PAM strength) for enAsCas12a activity at different sites used multiplicative penalty using data from similar large-scale screens of PAM libraries. In the case of RfxCas13d, penalties were recovered from taking the value of the reported log2(Fold-Change in expression) to the second power, vs. a perfectly complementary targeted mRNA reporter in their massively parallel screen for gRNA activity in the presence of mismatches. A missing value (rA-rC mismatch at position 15) was interpolated from the penalties of the rA-rC mismatches at positions 14 and 16. In the event of multiple sequential mismatches (two-in-a-row, three-in-a-row, etc.), the position-specific penalties for double- and triple- mismatches were used to calculate the CFD scores at those sites. If the off-target sites had <15 nt (nucleotide) identity as the intended target (<55% identity for RfxCas13d or <65% identity for enAsCas12a), the CRISPR effectors were considered effectively inactive at those sites.

### Design of Polyvalent Guide RNAs

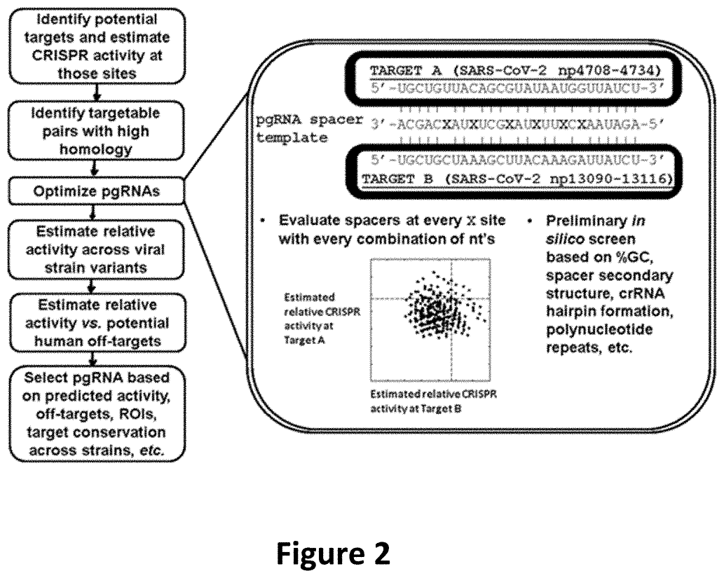

One example protocol for the design of polyvalent guide RNAs is summarized in FIG. 2, and implemented using MATLAB R2018a (Natick, Mass.) with the Bioinformatics Toolbox and the NCBI-BLAST+suite. Software for implementing the protocol are made available for non-commercial purposes upon request. To elaborate on each step of the protocol:

Step 1: Identification of Targets (‘protospacers’). For RfxCas13d, every 27 nt sequence along the Severe acute respiratory syndrome coronavirus 2 isolate Wuhan-Hu-1 complete genome was evaluated as a CRISPR target, also known as a ‘protospacer.’ For enAsCas12a, to recognized sufficiently by the enzyme protospacers must be located immediately downstream of a “Tier 1” protospacer adjacent motif (‘PAM’) (TTYN, CTTV, RTTC, TATM, CTCC, TCCC, and TACA) or a weaker “Tier 2” PAM (RTTS, TATA, TGTV, ANCC, CVCC, TGCC, GTCC, TTAC). Every 23 nt target targets sequences located immediately downstream of a Tier 1 or Tier 2 PAM sites were identified on either strand of the HIV-1 proviral reference genome and evaluated as a potential target/protospacer.

Step 2: Identification of Targetable Pairs with high homology. For each virus, every potential target was aligned to every other potential target, and pairs with >75% sequence identity (≥21 nt identity for Cas13d targets and ≥16 nt identity for Cas12a targets) identified. Those overlapping the SARS-CoV-2 poly(rA)-tail were removed from the list of potential pairs. For targeting the HIV provirus, exact target matches between pairs of sequences on the two long terminal repeat (LTR) regions were not considered (for reasons discussed below) unless they also formed a “target pair” with a segment between the two regions.

Step 3: Adaptation of pgRNA activity at pair sequences. For a given target pair, a pgRNA spacer template was generated complementary to the targets, using the location and sequences of the matching targets. Different ‘candidate pgRNA’ spacers were generated with all four potential nucleotides (rA, rU, rC, rG) at each of the sites of sequence divergence between the target pairs, i.e. 4n candidates for target pairs with n differences between sequence. A mismatch penalty (CFD score) between the candidates and each of the target pairs was calculated using the multiplicative approach (FIG. 2 right). For Cas13d, those with predicted relative activity (vs. the pgRNA candidate spacer's “on-target” or antisense sequence) ≥95% at both sites in the pair were kept for further evaluation, and those with <95% removed from the candidate list. For Cas12a, those with ≥20% relative activity (vs. the pgRNA spacer's “on-target” or complementary sequence) at both sites were kept for further evaluation. Candidate pgRNAs with homopolymer repeats (≥4 consecutive ‘rU’ or ≥5 consecutive ‘rG’, ‘rC’, or ‘rA’) were removed. Those with GC% <30% or >70% were also removed from consideration. The respective ‘direct repeat’ sequence for each crRNA (5′-ACCCCUACCAACUGGUCGGGGUUUGAAAC-3′ (SEQ ID NO: 2) for RfxCas13d and 5′-UAAUUUCUACUCUUGUAGAU-3′ (SEQ ID NO: 3) for enAsCas12a) sequence was appended 5′- to their pgRNA candidate spacers and the pgRNA secondary structures evaluated using the RNAfold function from MATLAB's Bioinformatic Toolbox. If the secondary structure of the direct repeat was perturbed by presence of the candidate spacer from its canonical structure, it was removed from consideration, as were those with secondary structure free energy in the spacer region lower than −5 kcal/mol.

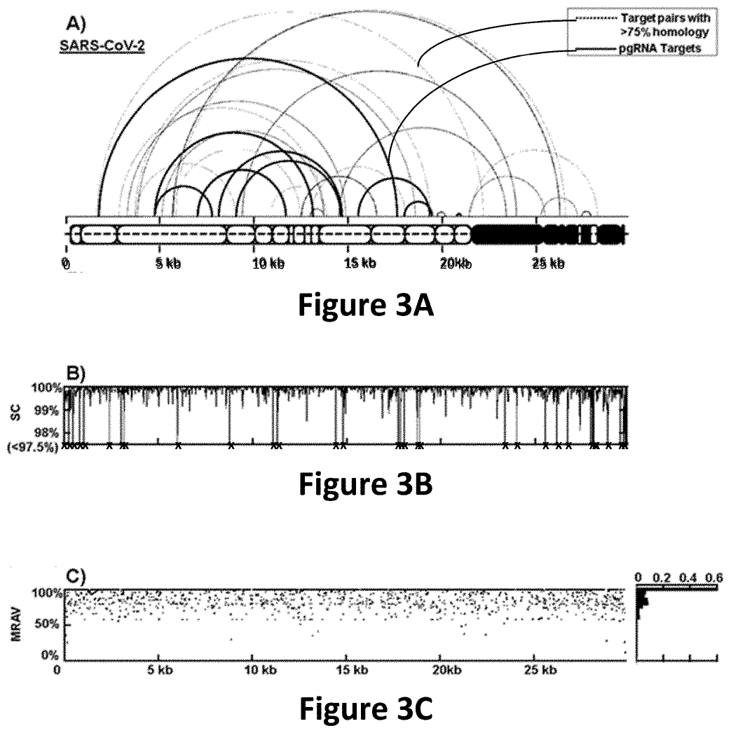

Step 4: Estimate relative CRISPR activity across clinical strains (SARS-CoV-2). Sequences of 942 SARS-CoV-2 clinical strain variants were downloaded from the Severe acute respiratory syndrome coronavirus 2 data hub (NCBI Virus, accessed Apr. 23, 2020) (48) as all the “complete” nucleotide sequences available at the time. The sequences were then each individually aligned to the Wuhan-1 reference strain using a Needleman-Wunsch global alignment, and for each potential target site (27 nt region) across the genome, the number and prevalence of unique variants were counted. In evaluating pgRNA candidates, if the minimum relative activity across variants (MRAV) for the candidate pgRNAs across all the sequenced SARS-CoV-2 strains was <95% at either target site, the candidates were flagged. Sequences with ambiguous sites or indels (because their effect on Cas13d and Cas12a are less well defined) were removed from the calculation. To evaluate sequence conservation and “conservation of targets” across the SARS-CoV-2 genome in general (i.e., FIG. 3B and FIG. 3C, resepctively), the most common target sequence was considered the “consensus” variant. The relative activity at each other unique variant was calculated using a gRNA for the consensus variant.

Step 5: Estimate relative activity at potential human off-targets. Candidate pgRNA spacers were aligned to the human genome for Cas12a (Genome Reference Consortium Human Build 38, GRCh38 human reference genome) or human transcriptome for Cas13d (GRCh38 human RefSeq transcripts) using a local nucleotide BLAST targeted for short sequences <30 nt (blastn-short). The region surrounding each hits to the human genome or transcriptome, to a total of 27 nt (the 27 nt protospacer for Cas13d and a 4 nt PAM+23 nt protospacer for Cas12a), were evaluated for a mismatch penalty score with its respective pgRNA candidates and, for Cas12a, the presence of a Tier 1 or Tier 2 PAM. While “off-target” interactions with the human transcriptome by Cas13d is not expected to have too detrimental of consequences compared to off-target genomic mutations by Cas12a, these unwanted interactions may titrate or dilute the activities of the Cas13d against the desired targets. For Cas13d, pgRNA spacer candidates with maximum predicted relative activity at any human transcript ≥10% were removed and, for Cas12a, those with maximum predicted relative activity at any site in the human genome ≥1% were removed.

Step 6: Selection of pgRNA based on additional functional criteria. At this stage, the RNA candidates have been screened for high relative activity at multiple viral targets and across clinical strains, low predicted activity at human “off-target” sites, and biophysical characteristics that suggest high overall CRISPR activity. The candidates can then be further refined by considering pgRNA targets located within specific genes or regions of interest (ROIs) that may be of clinical or functional significance, conservation of the targets/viral intolerance to mutations, and on-target activity prediction, which can be performed using several bioinformatic tools and methods available, prior to experimental validation.

### Design of Polyvalent Guide RNA Computer Implemented Code

One example computer implemented protocol for the design of polyvalent guide RNAs is s coded and made available at: https://github.com/ejosephslab/pgrna. This example code can be executed by a computing system such as a laptop, personal computer, or other device configured to read the code.

### Prevalence of pgRNA Target Pairs in Viral Genomes and pgRNA Candidates for Human-Hosted Viruses

All complete sequences of all RNA viruses with human, mammal, arthropoda, ayes, and higher plant hosts found in the NCBI Reference Sequence database were subjected to a brute force direct (nucleotide-by-nucleotide, no gaps) alignment for each of their 23 nt sequence targets to each other, considering only sequence polymorphisms at the same site. We considered only the (+) strand, as even for (−) and dsRNA viruses these sequences would match the vast majority of mRNA sequences. Only targets lacking polynucleotide repeats (4 consecutive rU's, rC's, rG's, or rA's) were considered viable targets. Targets derived from different segments or cDNAs of the same viral strain were considered together. In total: arthropoda (1074 viral species), ayes (111), mammal (496), higher plant/embrophyta (691), and human (89)-hosted viruses were considered. For human-hosted (+) ssRNA viruses or sequenced viral transcripts (59 in the RefSeq database), candidate pgRNA sequences for RfxCas13d were generated for each target pair found with predicted (monovalent) activity at both sites to be in the top quartile,²⁵ screened for biophysical compatibility (lacking polynucleotide repeats or significant predicted secondary structure in the spacer), and aligned to Genome Reference Consortium Human Build 38, GRCh38 human reference transcriptome) using a local nucleotide BLAST³⁴ search optimized for short sequences <30 nt (blastn-short). Only those with no hits (less than 15 nt homology out of 23 nt targets) to the human transcriptome and with predicted activity at both sites to be within the top quartile of all Cas13 activity for targets of that virus were considered viable pgRNA candidates.

### Estimation of SARS-CoV-2 Target Sequence Conservation

All complete SARS-CoV-2 genomic sequences available from the NCBI Virus database were downloaded on Nov. 23, 2020 (29,123 sequences). For each of the 205 target pairs possessing biophysically feasible pgRNA candidates, we aligned (no gaps) each target sequence to each genome to determine the closest matching sequence. Alignments containing ambiguous nucleotide calls were not included. Sequence variants were grouped together, with a minimum prevalence of 0.1%, with the fraction of hits by the most prevalent group being considered the sequence conservation reported.

### Construction of RfxCas13d for In Planta Expression

The DNA sequences of the plant codon optimized Cas13d-EGFP with the Cas13d from Ruminococcus flavefaciens (RfxCas13d) flanked by two nuclear localization signal (NLS) was amplified from plasmid pXR001 (Addgene #109049) using Q5 high fidelity of DNA polymerase (NEB). Similarly, overlap extension PCR was performed to amplify plant expression vector pB_35S/mEGFP (Addgene #135320) with ends that matched the ends of the Cas13 product so RfxCas13d expression would be under the control of 35S Cauliflower mosaic virus promoter. The PCR products were treated with Dpnl (NEB), assembled together in a HiFi DNA assembly reaction (NEB), transformed into NEB10b cells (NEB), and grown overnight on antibiotic selection to create plasmid pB_35S/RfxCas13. Successful clones were identified and confirmed by sequencing followed by transformation into electro-competent Agrobacterium tumefaciens strain GV3101 (pMP90).

### Construction of crRNA Expression Vector

Single stranded oligonucleotides corresponding to “monovalent”, non-targeting (NT), and “polyvalent” gRNAs were purchased from Integrated DNA Technologies (Coralville, Iowa), phosphorylated, annealed, and ligated into binary vector SPDK3876 (Addgene #149275) that had been digested with restriction enzymes XbaI and XhoI (NEB) to be expressed under the pea early browning virus promoter (pEBV). The binary vector containing the right constructs were identified, sequenced and finally transformed into Agrobacterium tumefaciens strain GV3101. Multiplexed expression of two crRNAs was achieved by ligating (annealed, phosphorylated) oligos for two individual crRNAs (hairpin+spacer) together with an internal 4 nt “sticky-end” and into SPDK3876 so both crRNAs would be expressed on a single transcript.

### Agroinfiltration of Nicotiana benthamiana (Tobacco) Leaves

In addition to pB 35S/RfxCas13 and the SPDK3876′s harboring gRNA sequences (TRV RNA2), PLY192 (TRV RNA1) (Addgene #148968) and RNA viruses TRBO-GFP (Addgene # 800083) were individually electroporated into A. tumefaciens strain GV3101. Single colonies were grown overnight at 28 degrees in LB media (10 g/L tryptone, 5 g/L yeast extract, 10 g/L NaCl; pH 7). The overnight cultures were then centrifuged and re-suspended in infiltration media (10 mM MOPS buffer pH 5.7, 10 mM MgC12, and 200 μM acetosyringone) and incubated to 3-4 hours at 28 degrees. The above cultures were mixed to a final OD600 of 0.5 for CasRX-NLS-GFP-pB35, 0.1 for PLY192 (TRV RNA1), 0.1 for RNA2-crRNAs and 0.005 for TRBO-GFP and injected into healthy leaves of five to six-week-old N. benthamiana plants grown under long-day conditions (16 h light, 8 h dark at 24° C.). A total of four leaves for each gRNA were infiltrated. Three days post-transfection, leaves were cut out and photographed under a handheld UV light in the dark, and stored at −80° C. before subsequent analysis.

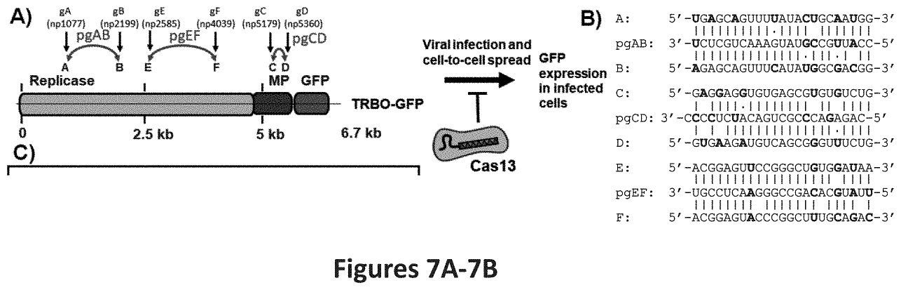

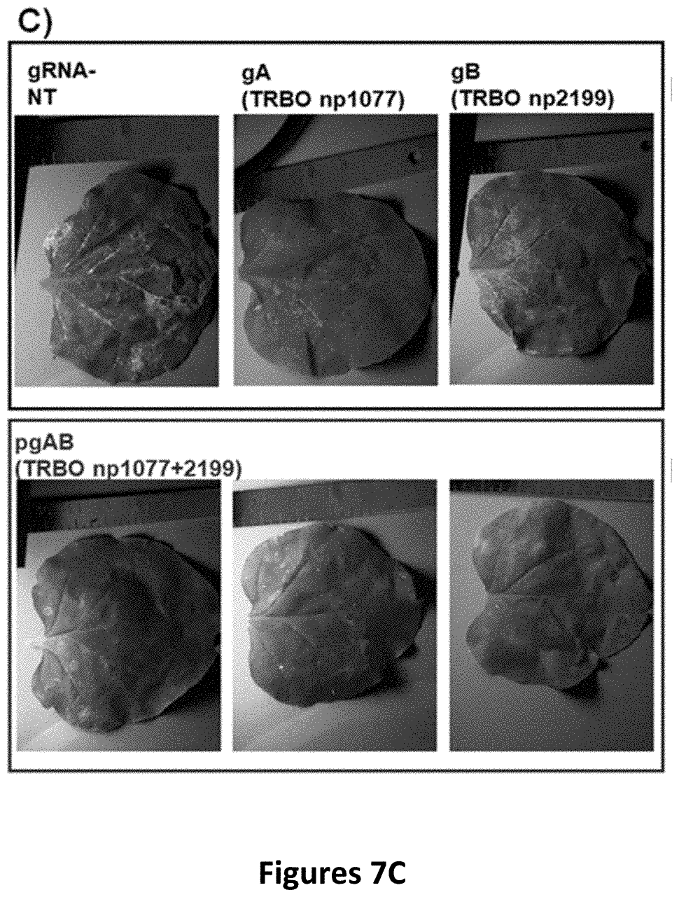

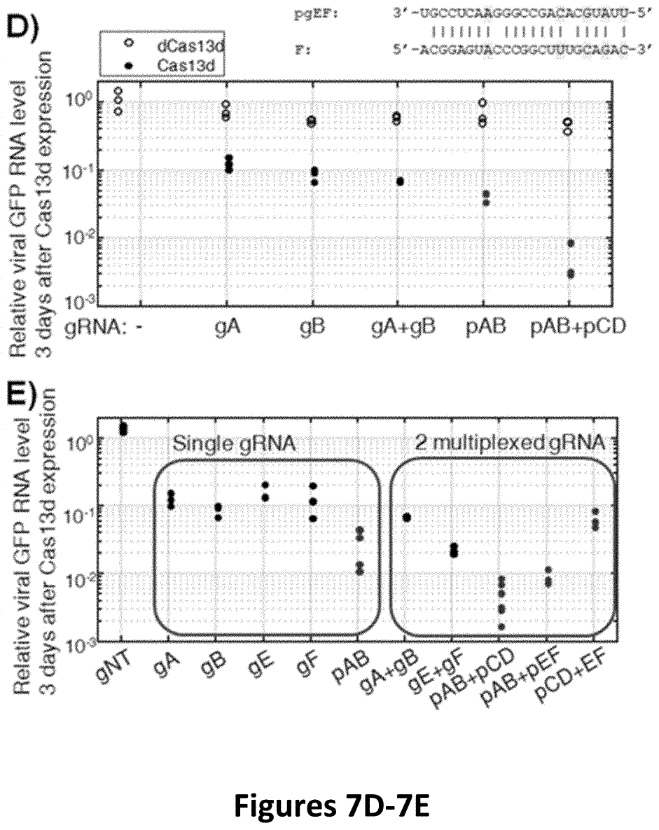

Referring now to FIG. 7A, the illustration depicts pgRNAs for RfxCas13d were designed to target pairs of sequences in the tobacco mosaic virus (TMV) variant replicon (TRBO-GFP) genome (left) with target sequences for monovalent (g; black) and polyvalent (pg; red) gRNAs labelled with arrows. (right) After infiltration of the replicon DNA and transcription, the (+) ssRNA virus will infectiously spread cell to cell in the leaf, the extent to which can be tracked by expression of a reporter protein (GFP). Viral spread is inhibited by TRBO-GFP-targeting RfxCas13d RNPs, providing a quantitative assay for antiviral activity by different gRNA designs. MP: movement protein. GFP: green fluorescent protein.

Referring now to FIG. 7B, the image displays pairs of targets in the TRBO-GFP for the different pgRNAs that had up to 30% (6 nt out of 23) divergence between sequences.

Referring now to FIG. 7C, leaves of N. bethamiana were infiltrated with a suspension of A. tumefaciens harbouring plasmids for the transient expression of RfxCas13d; one or two gRNAs (pgRNA, its two “monovalent” counterpart gRNAs, or a non-targeting (NT) gRNA, for example); and an expression cassette for replication-competent TRBO-GFP. Representative images of leaves illuminated under UV light three days after infiltration show the extent of viral spread by GFP expression. Viral spread is suppressed by Cas13 RNPs with gRNAs and strongly by Cas13 RNPs with pgRNAs, but not Cas13 RNPs with a non-targeting (NT) gRNA.

Referring now to FIGS. 7D-7E, these graphs depict quantitative reverse-transcription PCR (qRT-PCR) of leaf RNA after transient expression demonstrates that pgRNAs successfully inhibit viral spread in a higher organism better than their monovalent counterparts, at least as well as multiplexed monovalent gRNAs, and even better as multiplexed pgRNAs—reducing viral RNA levels by >99.5%. dCas13d: Catalytically inactive RfxCas13d mutant. (p-values for two-sided T-test; N=4 leaves each).

### Quantitative RT-PCR

Total RNA was extracted from infiltrated leaves using RNeasy Plant Mini Kit (Qiagen) and the yield was quantified using a nanodrop. A total of lug RNA from control (NT gRNAs) and experimental samples were used for DNase I treatment (Ambion, AM2222) followed by reverse transcription using a poly-dT primer and the Superscript III First Strand cDNA Synthesis System for RT-PCR (Invitrogen). Quantitative PCR was performed on Quant studio 3 Real-Time PCR System from Applied Biosystem using iTaq PowerUP™ SYBR Green pre-formulated 2× master mix (Applied Biosystems). Relative expression levels based on fold changes were calculated using the ddCT method. Cycle 3 GFP mRNA expression levels from the TRBO-GFP replicon were normalized against transcripts of the tobacco PP2A. The samples were performed in three biological replicates.

### Cas13 Collateral Activity Assays

Initial screens were performed using synthetic dsDNA (˜300 bp) containing a T7 promoter located upstream of a specific target sequence derived from either SARS-CoV-2 (FIG. 3C and S7) or human CD46 transcript sequences (FIG. 3B) in two steps as follows: 1 μl Leptotrichia wadeii Cas13a (LwaCas13a) enzyme (106 ng; Molecular Cloning Laboratories, South San Francisco, Calif., US) was preincubated with each pre-synthesized gRNA [0.25 uM; Integrated DNA Technologies, Coralville, Iowa, US (IDT)] in a total volume of 5 μl for 10 min at room temperature, followed by the addition of 16 μl of synthetic dsDNA template (Twist Biosciences, South San Francisco, Calif., US) at varying concentrations (4.0×10⁵ cp/μl, 4.0×10⁷cp/μl, or 4.0×10⁹ cp/μl at final concentration for SARS-CoV-2 targets and 1.0×10⁹cp/ul for CD46 targets). A master mix containing 0.5 μl of T7 RNA polymerase [New England Biolabs, Ipswich, Mass., US (NEB)], 1 μl of 25 mM rNTPS (at equal ratios of rATP, rUTP, rGTP, rCTP; NEB), 0.23 μl 1 M MgCl2 (Invitrogen ThermoFisher, CA US), 0.5 ul HEPES (Invitrogen ThermoFisher, CA US), 0.63 μl of RNAseH inhibitor (NEB), 1.56 μl RNAse Alert Reporter (IDT), and 0.58 ul of nuclease-free water (Invitrogen) were assembled on ice and 4 μl added to the mixture containing the DNA template and preincubated Cas13 RNP. 25 μl of each preassembled reaction was added to a 384 well plate (Black/Clear Bottom) and loaded into a preheated fluorescence microplate reader (Promega GloMax Explorer) at 37° C. Data readouts were collected every 5 min for 1 hr at an excitation peak at 480 nm and an emission peak at 520 nm.

Specificity of Cas13 collateral activity was evaluated using dsDNA fragments that were not complementary to the gRNAs being tested to confirm that activation of collateral activity as well as human universal RNA (10 tissues) (Invitrogen ThermoFisher, CA US), and total human lung RNA (Invitrogen ThermoFisher, CA US), was also used at 1 and 3 ug, respectively per reaction.

### SHERLOCK-Type Viral Detection Reactions

Heat-inactivated SARS-CoV-2 RNA from respiratory specimens, deposited by the Centers for Disease Control and Prevention, was obtained through BEI Resources, NIAID, NIH: Genomic RNA from SARS-Related Coronavirus 2, Isolate USA-WA1/2020, NR-52285 (American Type Culture Collection (ATCC) VR-1986HK). In a SHERLOCK-type reaction, 1 μl of heat-denatured SARS-CoV-2 (350,000 copies total) was reverse transcribed using the High Capacity cDNA Reverse Transcription Kit (Thermo Fisher Scientific) with 3.4 μl of primer (0.5 μM) in a final volume of 16 μl and PCR-amplified by the addition of 2 μl of reverse and forward target primers (2 μM) and 20 μl of 2× OneTaq Master Mix (NEB) in a final volume of 40 μl under standard thermocycler conditions (2 min at 95° C., followed by 35 cycles of 30 s at 95° C., 30 s at 49° C., and 30 sec at 68° C., followed by a final extension of 5 min at 72° C.). PCR cDNA targets were then combined accordingly, and serial dilutions were made such that the final concentrations of the starting SARs RNA material in SHERLOCK reaction was adjusted to either 400, 40, or 4 copies per μl for each target. SHERLOCK reactions were performed as described early using candidate pgRNAs and their monovalent counterparts in the presence of none (background), one, two, or four cDNA targets per reaction. SHERLOCK reactions in the absence of guide RNA were also evaluated and resulted in equivalent background signals produced from no RNA template controls.

### In Vitro Transcription of Cas9 gRNAs

Single guide RNA (sgRNA) was synthesized by using the EnGen sgRNA synthesis Kit (NEB, New England Biolabs, Ipswich, Mass., United States) following standard protocols. DNA oligos (IDT) were designed to contain a T7 promoter sequence upstream of the target sequences with an initiating 5′- d(G), as well as overlapping tracrRNA DNA sequence at the 3′ end of the target. The sgRNA was purified using Monarch RNA Cleanup Kit (NEB) and quantitated using standard protocols.

### Duplex gRNA Generation

Duplex CRISPR gRNAs (cRNA:tracrRNA) was generated by hybridizing synthetic RNA oligos listed in Table S9 to a universal synthetic tracer RNA oligo (IDT). To hybridize oligos, equal molar concentration of oligos were combined in IDT duplex buffer to a final concentration of 10 uM. Reactions were heated to 95° C. for 2 min and allowed to cool to room temperature prior to the reaction assembly.

### Cas9 Cleavage Reactions

Cas9 Nuclease from S. pyogenes (NEB) was diluted in 1× NEB Buffer 3.1. prior to the reaction assembly. Cas9 cleavage activity was performed using either PCR-amplified targets, whole plasmid, or hybridized DNA oligos containing desired targets using standard methods. Briefly, Cas9 was preincubated with either a sgRNA or duplex gRNA (crNA:tracRNA) for 5 min at equal molar concentrations in 1× NEB Buffer 3.1 (NEB) in a volume total of 10 ul. Reactions were incubated for 5-10 min at room temperature. Target DNA was then added to the reactions, NEB Buffer 3.1 was added back to a final concentration of 1×, and nuclease-free water was added bringing the final volume to 20 ul. The final reaction contained 100 nM Cas9-CRISPR complex and 10 nM of target DNA. Similar reactions without the addition of gRNAs to Cas9 were used as a control for uncut DNA. Reactions were incubated at 37° C. for 1 hour, followed by the addition of 1 unit of Proteinase K and further incubation at 56° C. for 15 min. Reactions were stopped by the addition of one volume of purple Gel Loading dye (NEB).
Fragments were separated and analyzed using a 1.5% Agarose gel in 1×TAE and 1×SYBR Green 1 Nucleic Acid Gel Stain (Thermo Fisher Scientific; Waltham, Mass.), and fluorescence was photographed and measured (Amersham™ Imager 600; GE Life Sciences, Piscataway, N.J., United States).

### Results nd Discussion

### Similarities and Differences in the Design Criteria for gRNAs Used for Precision Gene Editing and Those Used for CRISPR Antivirals

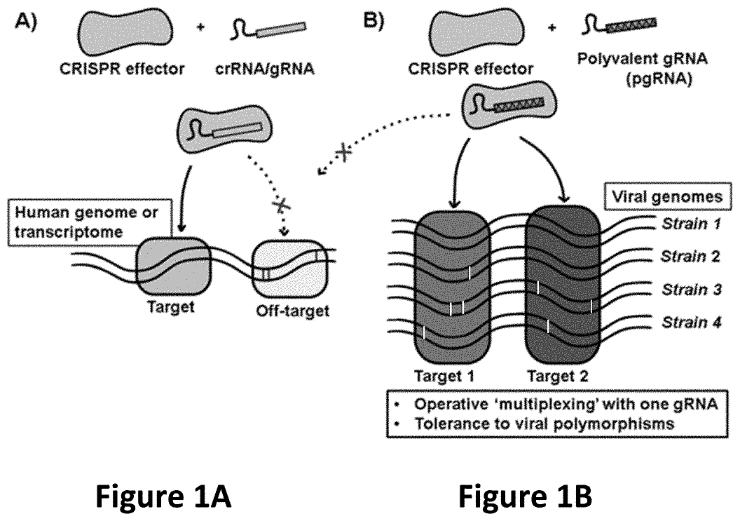

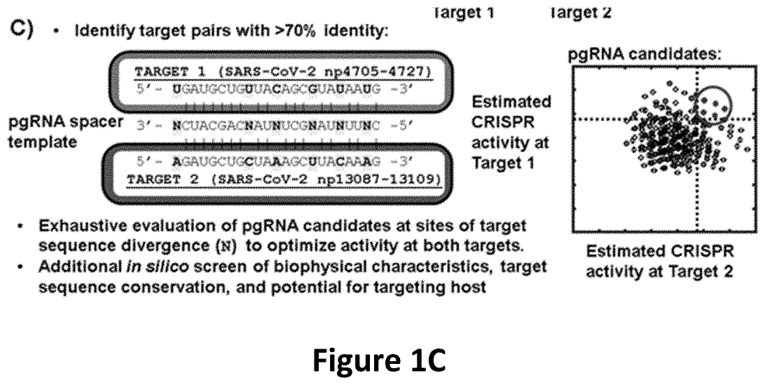

Despite significant differences in the goals and desired outcomes between CRISPR precision gene editing and CRISPR antivirals as illustrated in FIG. 1, there are some primary objectives in the design of targeting sequence of the gRNA spacer sequences shared by both applications. In particular, CRISPR activity at the desired target is maximized by identifying spacer sequences with no or weak internal secondary structures, moderate GC content (GC %, between ˜30%-70%), avoidance of polynucleotide repeats that may inhibit gRNA expression, and avoiding chromatin or occluded targets. Recent bioinformatics analyses have revealed additional sequence contexts and features that may be used to predict spacer sequences with maximized on-target CRISPR activity.

In the case of precision gene editing as shown in FIG. 1A, avoidance of CRISPR activity at any unintended or ‘off-target’ site is of paramount importance to prevent unwanted genetic mutations. When some flexibility exists in the choice of a specific target (the mutational knockout of a gene, for example), this is achieved by designing gRNA spacers targeted to sites with few other similar genomic sequences, or is otherwise performed by increasing the specificity of the CRISPR effectors, that is, limiting the tolerance of CRISPR effector for any mispairs between the spacer and the target. Increasing specificity of CRISPR systems for gene editing applications has been the subject of significant efforts, from structure-based engineering and directed evolution of the CRISPR effectors themselves to the destabilization or fine-tuning of spacer-target interactions to limit activity at sequences that are similar but imperfect matches to the desired target.

In contrast, for CRISPR antivirals as shown in FIG. 1B, avoidance of activity with human genomic or transcriptome must be balanced against a requirement for tolerance to sequence heterogeneities across viral targets. In antiviral applications, of paramount importance is the prevention of mutagenic escape—the loss of CRISPR antiviral activity as a result of heterogeneity across clinical strains or viral families at the target site, or as a result of non-inactivating mutations that might occur after mutagenic repair of CRISPR degradation at the target. As mentioned above, during antiviral applications, these challenges have typically been addressed by simultaneously introducing multiple gRNAs (up to six) to target different regions of the viral genome, limiting the possibilities for mutagenic escape, and targeting regions of high sequence conservation or functional importance where mutations might not be well tolerated. Currently, the design of gRNAs for CRISPR antivirals relies on the computational tools used for precision gene editing, which may lead to sub-optimal antiviral outcomes.

Referring to FIG. 1C, the graph displays an example protocol for designing pgRNAs: aftertarget pairs with >70% homology have been identified in the same viral genome, the nucleotides at positions where the sequence between the two targets differ are chosen to minimize potential reductions of activity at the different sites by determining which mismatch- and position-specific mispairings are best-tolerated by the CRISPR effector.

### Design Principles for Polyvalent gRNAs (pgRNAs)

We hypothesized that, if we could match target sequences within a viral genome to other targets on the same viral genome with some shared sequence homology, a single gRNA spacer sequence could be adapted to maximize CRISPR activity at both targets; this is, in effect, the opposite as what is performed during gRNA design for precision gene editing. The development of “polyvalent” gRNAs—with one spacer able to target multiple protospacers—would have multiple advantages for CRISPR antiviral applications: operative “multiplexing” with fewer components, limiting the potential for viral escape, and increasing the effective number of potential “targets” a CRISPR effector could recognize in viral detection applications. This approach could exploit the myriad of validated tools that are currently used to predict and minimize off-target activity to instead maximize the predicted activity at both those sites. However, because of the differences in the objectives of current gRNA design tools, polyvalent gRNAs would normally be algorithmically rejected, so new approaches are necessary.

The design of polyvalent gRNAs or pgRNAs relies on exploiting known tolerances of CRISPR effectors for mismatches between gRNA and the target to maximize activity at multiple viral sites. These tolerances exhibit a strong dependence on both the type of mismatch (what nucleotides are incorrectly paired) and the position of the mismatch(es) along the target, and vary not only by type of CRISPR effector but across homologues of the effector derived from different species.

Careful and systematic studies have been performed to better predict and minimize the propensity of “off-target” effects gene editing; for the design of pgRNAs, we can use these same studies to instead attempt to maximize activity of a single gRNA at multiple viral sites. A metric to score the relative propensities of a CRISPR effector at a site that does not perfectly match its target that is both powerful and simple-to-implement uses a Cutting Frequency Density (CFD) matrix to estimate the penalty or relative decrease in CRISPR activity at off-target sites as a result of each difference in sequence between the target and that site. This approach is described in more detail in the Materials and Methods section. The CFD matrix consists of the mismatch-and position-specific penalties that have been derived from massively parallel characterizations of off-target CRISPR activity, and for each expected mispairing between the gRNA and the off-target site, these penalties are multiplied together to obtain a final score or relative expected CRISPR activity at that site. CFD scores in precision gene editing are used to reject gRNAs which may exhibit high activities at multiple sites in a targeted genome.

The design of pgRNAs can use CFD scores as an example metric for increasing predicted activity at multiple viral sites based at least in part on the following approach as shown in FIG. 2: (i) first potential target sites on a viral genome are identified and matched those with sequence similarity (e.g., >75% identity); (ii) the positions of sequence differences between the pairs are located; (iii) a “template” pgRNA spacer is generated that is complementary to the shared nucleotide sequences of the targets, and from the template “candidate” pgRNA spacers with different nucleotides at the positions of sequence divergence are created; (iv) the different candidates are then scored according to the CFD at both targets; (v) then, if a candidate receives a passing score (expected relative activity at both sites greater than a threshold level), a further analysis of those candidates is performed. This further analysis includes scoring the potential off-target activity at the human genome or transcriptome, and determining the minimum relative activity across variants (MRAV) by calculating CFD for the pgRNA candidates at each site across different clinical viral strains (tolerance to sequence heterogeneities). In this way, our gRNA design algorithm focuses explicitly on the major design considerations (multiplexing/preventing escape; tolerance for clinical variation/viral sequence heterogeneity) for CRISPR antivirals applications.

For instance, FIG. 2 provides one example design protocol for polyvalent guide RNAs (pgRNAs) in accordance with the present disclosure. Briefly, after pairs of targetable sequences in the viral genome with large fractions of identical sequence (e.g., ≥75%) are identified, a pgRNA spacer template is generated (right). For pairs with n sites where the sequence differs, 4n candidate pgRNA spacers are generated with every possible combination of nucleotides at those n sites, which are then evaluated for sufficient predicated relative activities at both target pairs using a Cutting Frequency Determination (CFD) score. They are then screened in silico for acceptable biophysical properties known to affect CRISPR activity (secondary structure, GC %, etc.). Those pgRNA candidates with acceptably high relative activity across all clinical strain variants and acceptably low predicted activity at potential off-target sites with the human genome/transcriptome can then be further screened for additional criteria (targeting specific genes or regions of interest (ROIs), for examples) and evaluated using additional gRNA design tools or validated experimentally.

More particularly, candidate pgRNAs were also evaluated in silico for biophysical characteristics, like GC %, secondary structure free energy, and the ability of the ‘direct repeat’ segment of the gRNA to form (which is essential for CRISPR activity) as preliminary indicators for a high likelihood of strong on-target activities. We note that the CFD calculated in the way described above provides an estimate of CRISPR activity at the viral sites relative to a hypothetical target with a sequence perfectly complementary to the pgRNA spacer: this allows us later to integrate our pgRNA design algorithm into other computational tools that predict CRISPR activity at on-target/perfectly matched sequences.

### pgRNAs for RfxCas13d Against SARS-CoV-2 Genomic RNA

We first sought to determine if we could generate novel pgRNAs for RfxCas13d that could be expected to exhibit high activity at multiple viral targets in SARS-CoV-2, the etiological agent of the infectious respiratory illness human COVID-19, while maintaining minimal activity with potential human off-targets (FIG. 3). We made this choice because of the broad tolerance for mismatches exhibited by RfxCas13d; its lack of PAM or sequence requirements outside the protospacer; and its recent demonstrated antiviral activity in human cells against ssRNA virus SARS-CoV-2. The large SARS-CoV-2 genome has 29,876 potential 27 nt segments that can be recognized by an antisense 27 nt spacer of the RfxCas13d gRNA. Antiviral activity of Cas13 was increased by multiplexed targeting, using up to four gRNAs targeting different viral sites. pgRNAs with high activity at multiple sites could therefore dramatically increase their effectiveness and power without increasing the complexity or components of the system.

For instance, FIG. 3 provides on example for identifying (A) targets with high identity and pgRNA targets, (B) sequence conservation and (C) the lowest relative predicted CRISPR activity across clinical strains of ssRNA virus SARS-CoV-2. FIG. 3A illustrates pairs of 27 nt Cas13d targets along the SARS-CoV-2 genome that were identified as having ≥75% identity (at least 21 out of 27; gray dotted lines). Pairs where pgRNAs could be designed with relative predicted activity at both sites >95%, and predicted activity at any similar elements of the human transcriptome <10%, were labelled in red. (below) Map of the SARS-CoV-2 genome, with ORFs labelled by as rounded rectangles; individual ORFs with multiple protein products (e.g., the ORFlab polyprotein) labelled as blocks of the same colour for each product. FIG. 3B illustrates sequence conservation of 27 nt targets across 942 sequenced clinical samples of SARS-CoV-2, showing targets located every 14 nt apart for clarity. FIG. 3C illustrates minimal relative activity across variants (MRAV) predicted for Cas13a activity using a crRNA targeted to the “consensus” target sequence (most common sequence) across all the 942 sequenced clinical samples of SARS-CoV-2, relative to on-target activity (showing targets located every 14 nt apart for clarity). (right) Histogram showing that ˜60% of crRNAs targeting the consensus sequence exhibit >95% relative activity across all clinical strain variants.

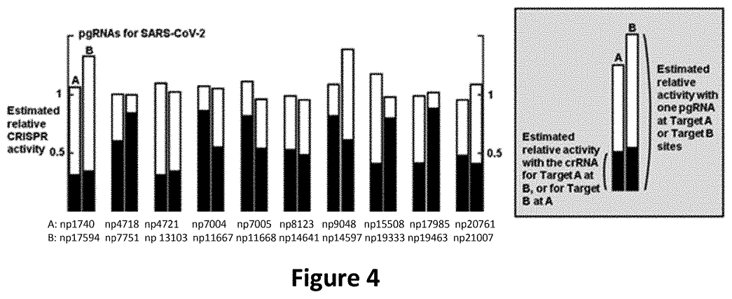

We first identified 81 pairs of target sites along the SARS-CoV-2 reference genome that had >75% (21/27) nt sequence identity (FIG. 3A and Table 1). Prior to performing the pgRNA adaptation, if we simply considered the expected activity of the gRNAs for one target at its “paired” sequence, using the mismatch penalty/CFD score we would expect only 43% median relative CRISPR activity at their other paired site. After our pgRNA adaptation, 249 candidate pgRNA spacers were identified total across 17 of the 81 pairs, where the predicted relative CRISPR activity is expected to exceed 95% at each site. Of the pairs with those active candidates, 10 pair sites had pgRNA candidates with <10% maximum predicted activity to elements of the human transcriptome (FIG. 3A and FIG. 4.

TABLE 1
Statistical analysis of in silico generation
and characterization of pgRNA candidates.

| | | | HIV-1 |
|---|---|---|---|
| | | SARS-CoV- | proviral dsDNA |
| | X | 2 ssRNA genome | genome |
| CRISPR effector: | — | RfxCas13d | enAsCas12a |
| Viral genome size: | — | 29903 | 9719 |
| Total # potential target | — | 29876 | 2834¹ |
| sites | | | |
| # target pairs with >X | 75% | 81 | 56² |
| homology: | | | |
| | 95% | 17 | — |
| # unique target pairs | 20% | — | 6 |
| with pgRNA candidates >X | | | |
| activity: | | | |
| # pgRNA spacer | 95% | 249 | — |
| candidates with >X activity at | | | |
| both each target site: | | | |
| | 20% | — | 156 |
| # unique target pairs | 10% | 10 | — |
| with active pgRNA candidates | | (transcriptome) | |
| and <X activity vs. human | | | |
| genome/transcriptome: | | | |
| | 1% | — | 5 (genome) |
| Total # unique target | — | 5 | 5 |
| pairs with pgRNA candidates | | | |
| passing in silico screen: | | | |
| Total # pgRNA | — | 25³ | 47 |
| candidates passing in silico | | | |
| screen: | | | |

¹Number of targets on both strands to the immediate 5′- of a Tier 1 or Tier 2 enAsCas12a PAM.
²177 pairs, including exact matches located within the long terminal repeat (LTR) regions of the HIV-1 provirus.
³125 candidates identified with <10% activity vs. human transcriptome and >95% activity targeting the reference strain sequence; 25 candidates identified with <10% activity vs. human transcriptome and >95% activity across clinical strains.

The viral targets sites for CRISPR effectors are often chosen based not only on the gene product encoded but also by conservation of nucleotide sequence across clinical strains or related viral families. However, based on the differential ability of CRISPR effectors to recognize and degrade targeted sequences in spite of mismatches between the gRNA and the protospacer, we endeavoured to quantify the “conservation of targets” (rather than sequence, per se) as potential target sites where CRISPR effectors may be highly active across strains regardless of the presence of certain sequence variations. To evaluate the “target conservation” at each of these candidate pgRNA spacers, first we aligned the 942 sequenced viral genomes from clinical samples to the reference Wuhan-1 sequence and characterized their variability. Approximately 50% (50.07%) of the target sites possessed sequence identity, or perfect sequence conservation (SC), across all 942 samples over the entire 27 nt range (FIG. 3B); 96% of target sites had SC across at least 99% of the samples. Of the 50% of sites that were not identical, however, 25% of those sites were expected to exhibit a minimum relative activity across variants (MRAV) of >95% activity relative to a gRNA targeting the consensus (most common) sequence (FIG. 3C right). 80% were expected to exhibit an MRAV of at least 75%, with a median MRAV across targets with imperfect SC of 85.6%, and 1.56% with predicted MRAV of <50%. Of the 10 paired sites that were targetable by the pgRNAs, 5 of those pairs had pgRNA candidates that maintained expected minimum relative activity of greater than 95% across the 942 clinical strains at both sites. Those are the top candidate pgRNA spacers reported in Table 2.

Genetic targets for detection and inactivation SARS-CoV-2 virus have largely been focused on the highly conserved genes for nucleocapsid protein N and the gene for the RNA-dependent RNA polymerase (RdRP), which is essential for viral replication. Interestingly, the top candidate pgRNA spacers each have two target sites localized across ORF lab, which encodes a large polyprotein later processed into smaller nonstructural proteins (nsp), several of which are important for viral replication. Two of the pairs have one target within the segment of ORF lab that encodes the RnRP. The results presented here demonstrate that pgRNAs can be designed for RfxCas13d that simultaneously are expected to exhibit high relative activity at multiple (essential) target sites on the SARS-CoV-2 genome for which “target conservation” is high, while minimizing expected interactions with the human transcriptome.

TABLE 2
pgRNA spacer candidates for RfxCas13d against
the SARS-CoV-2 ssRNA genome¹

| | | | Maximum |
|---|---|---|---|
| | | | relative |
| | | | predicted |
| | | | pgRNA |
| pgRNA | Target A² (ORF/ | | activity |
| spacer sequence²; | product); | Target B/C | at BLASTn |
| Target A antisense; | Relative | (ORF/product); | hits to human |
| Target B antisense. | Activity (Δ)³ | Activity (Δ) | transcriptome³ |
| 5′-UAACCAUUGUUCGCUG | np4718 | np7751 | (no BLASTn |
| UAACAGUAUCA-3′ (SEQ | (ORF lab/nsp3); | (ORF lab/ | hits) |
| ID NO: 4); | 1.002 (+0.396) | nsp3); 0.996 | |
| 3′-AUUGGUAAUAUGCGAC | | (+0.155) | |
| AUUGUCGUAGU-5′ (SEQ | | | |
| ID NO: 5); | | | |
| 3′-CUUGGUAAGAAGUGAC | | | |
| AUUGUGAUAGU-5′ (SEQ | | | |
| ID NO: 6). | | | |
| 5′-AGAUAAACGUUCUAUG | np4721 | np13103 | (no BLASTn |
| CUUUAACAGCA-3′ (SEQ | (ORF lab/nsp3); | (ORF lab/ | hits with |
| ID NO: 7); | 1.097 (+0.779) | nsp10); 1.021 | >15/27 nt |
| UCUAUUGGUAAUAUGCGAC | | (+0.669) | aligned) |
| AUUGUCGU-5′ (SEQ ID | | | |
| NO: 8); | | | |
| 3′-UCUAUUAGAAACAUUC | | | |
| GAAAUCGUCGU-5′ (SEQ | | | |
| ID NO: 9). | | | |
| 5′-ACAUUGUUGGCAAGUU | np8123 | np14641 | (no BLASTn |
| CAGCUACUGUA-3′ (SEQ | (ORF lab/nsp3); | (ORF lab/RNA- | hits with |
| ID NO: 10); | 0.988 (+0.458) | dependent RNA | >15/27 nt |
| 3′-UGUAAGAAACGUUCAA | | polymerase); | aligned) |
| GUCGAAGACGU-5′ (SEQ | | 0.955 (+0.469) | |
| ID NO: 11); | | | |
| 3′-UGUAACAAUCAUUCAC | | | |
| GUCGAUGACUU-5′ (SEQ | | | |
| ID NO: 12). | | | |
| 5′-AUAUAGUAGUAGAUUA | np9048 | np14597 | (no BLASTn |
| ACCAGAGCAUC-3′ (SEQ | (ORF lab/nsp4); | (ORF lab/RNA- | hits with |
| ID NO: 13); | 1.061 (+0.460) | dependent RNA | >15/27 nt |
| 3′-UAUACCAUGACCGAAU | | polymerase); | aligned) |
| GGUCUUCGUAG-5′ (SEQ | | 1.350 (+0.549) | |
| ID NO: 14); | | | |
| 3′-UAGAUCAUUAUCUAAU | | | |
| GGUCUUCGUCG-5′ (SEQ | | | |
| ID NO: 15). | | | |
| 5′-UAAAUUGCAACCUGUC | np17985 | np19463 | (no BLASTn |
| AUAAACGUGUC-3′ (SEQ | (ORF lab/ | (ORF lab/3′-to- | hits) |
| ID NO: 16); | helicase); | 5′ exonuclease); | |
| 3′-AUUUAACGUUGAACAG | 0.988 (+0.568) | 1.019 (+0.137) | |
| UAUUUCCAGAG-5′ (SEQ | | | |
| ID NO: 17); | | | |
| 3′-AUUUAACGUUGCACAA | | | |
| UAUGUGCAUCG-5′ (SEQ | | | |
| ID NO: 18). | | | |

¹pgRNAs have >95% predicted relative activity at both targets; <10% predicted relative activity at hits to human transcriptome; and >95% predicted relative activity across all (948) clinical rains
²Underlined at sites where Target A and Target B/C sequences diverge
³Labelled according to np (nucleotide position) of central nucleotide of 27 nt protospacers, (according to SARS-CoV-2 Wuhan-1 strain). nsp: nonstructural protein
⁴Δ: Increase in predicted CRISPR activity by using pgRNA at target A or B, compared to using gRNA for target A at target B (or vice versa)
⁵Nucleotide BLAST targeted for short (<30 nt) sequences vs. GRCh38.p12 RefSeq transcripts.

### pgRNAs for enAsCas12a Against HIV-1 Provirus

To determine whether we could generate pgRNAs for against a dsDNA virus, we targeted the HIV-1 provirus using a Cas12a effector (FIG. 4a). Recent reports indicate that Cas12a was more effective in curing cells of the HIV provirus and preventing mutagenic escape than Cas9, as a result of different mutational patterns induced by Cas12a DSBs better able to inactivate the virus compared to Cas9 DSBs. Because the resulting mutations are still subject to variations, we hypothesized that use of pgRNAs might further increase the effectiveness of these approaches, as multiplexing or targeting multiple viral locations simultaneously suppresses mutagenic escape and increases the probability of generating a disabling mutation.

However, there are additional challenges for targeting the HIV-1 proviral genome using Cas12a. The HIV-1 proviral genome is smaller (9719 bp) than the SARS-CoV-2 genome and, while both strands of the dsDNA could be targeted, unlike Cas13, Cas12a can only target sequences positioned immediately downstream a protospacer adjacent motif (PAM') that is recognized by the enzyme itself rather than the gRNA. Even with engineered enAsCas12a, which is able to recognize a larger number of PAMs than the native enzyme, strong PAM sequences (Tier 1 or Tier 2) able to activate robust endonucleoltyic activity only appear on average every 1 in 16 bp. Additionally, off-target DSBs on the human genome hold the potential for significant deleterious consequences, so we require pgRNAs with even less potential for accidental targeting of human off-targets than Cas13.

In particular, FIG. 4 illustrates the estimated relative RfxCas13d CRISPR activities of SARS-CoV-2 pgRNAs after adaptation for activity at multiple target sites. (black bars) Estimated activities of RfxCas13d using a gRNA for Target A at the Target B sequence, or vice versa. (white bar) Estimated activities at the two target sequences after adaptation (see FIG. 2). (below) The nucleotide position (np) of Target A and Target B for each pgRNA, labelled by the central nt of a 27 nt Cas13d pgRNA spacer.

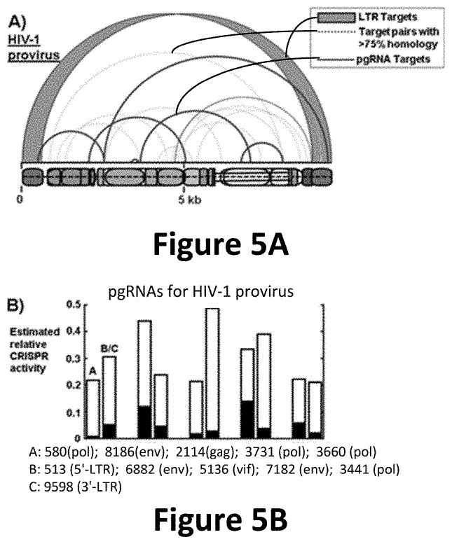

With these considerations taken into account, we identified 177 target sites next to Tier 1 or Tier 2 PAMs of enAsCas12a in the HIV-1 proviral genome that shared >75% homology across 23 bp targets (FIG. 5 and Table 1). 112 of the 177 pairs were identical targets localized to the long terminal repeat (LTR) regions that flank protein-coding regions (FIG. 5). Because HIV-1 appears highly tolerant to mutations within the 5′- and 3′- LTRs, we did not consider these pairs for further analysis unless one member targeted the protein-coding region. Of the remaining 65 pairs, if as before we simply estimated the activity of the gRNAs for one target at its “paired” sequence prior to performing the pgRNA adaptation, we would expect only 3.8% median CRISPR activity at those paired sites. After pgRNA adaptation, we identified 156 candidate pgRNAs able to target six of the pairs and one set of three targets (two identical sites in the LTRs and one site in the protein-coding region) with predicted relative activity >20%, which was previously used as a milestone for Cas12a high activity, and satisfactory biophysical parameters. Of those, we were able to identify pgRNAs for 5 of those 6 pairs/sets where predicted off-target activity (on homologous sites in the human genome) was <1%, including for the pgRNA with high activities at three viral targets (FIG. 5). Several example pgRNA candidates displaying higher on-target activities, are reported in Table 3 below, although up to 47 candidates passing the in silico screen were identified (Table 1). One example method for calculating the on-target activity score can include applying an available algorithm (such as the Broad institute's sgRNA designer https://portals.broadinstitute.org/gpp/public/analysis-tools/sgrna-design for Cas9) to calculate activity. In practice, we only align targets or try to find target pairs where the predicted on-target activity at one or both sites is in the top quartile of all potential targets in a viral genome, but that can be limiting to the top X %.

For instance, FIGS. 5A and 5B illustrate one example of identifying sets of 23 bp enAsCas12a targets along the HIV-1 proviral genome with ≥75% identity were identified (at least 16 out of 21; gray dotted lines). FIG. 5A depicts pairs where pgRNAs could be designed with relative predicted activity at both sites >20%, and predicted activity at any similar elements of the human genome <1%, were labelled in red. (below) Map of the HIV-1 proviral genome, with ORFs labelled by as rounded rectangles; individual ORFs with multiple protein products labelled as blocks of the same colour for each product. Long terminal repeats (LTR) regions that flank the protein-coding regions are labelled in blue. FIG. 5B depicts estimated relative CRISPR activity of the pgRNAs at multiple HIV-1 targets. Below, sites of the targeted sets, labelled from the first protospacer position nearest the PAM site. See Table 3 and FIG. 4 for legend.

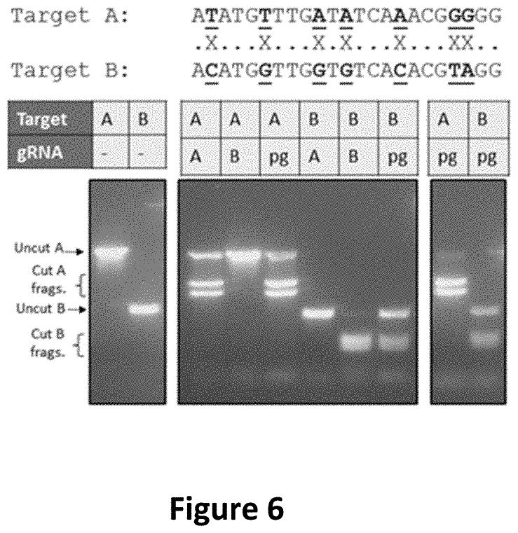

To further validate proposed example implementations, a Cas9 pgRNAwas designed for two virally-derived targets. As shown in FIG. 6, Two potential target sequences from Tobacco Rattle Virus (TRV) differ at 7 out of 23 nucleotide sites (˜30% divergence). We algorithmically designed a single pgRNA (pg) for CRISPR effector Cas9 to degrade (cleave) viral cDNA at both the sites. PCR fragments of the TRV viral cDNA containing the target sequences were incubated with the CRISPR ribonucleoprotein (RNP) complexes, then the products separated by size using agarose gel electrophoresis. While the Cas9 RNPs with gRNAs specific to Target A otherwise exhibit no activity at Target B, and the Cas9 RNPs with gRNAs specific for Target B exhibit no activity at Target A as well, the Cas9 RNPs with the pgRNA that we computationally designed exhibits significant activity at both viral targets.

The crRNA spacer sequences and target sequences for the above data are provided below:

| | crRNA A |
|---|---|
| (SEQ ID NO: 19) | |
| | ACAUGGUUGGUGUCACACGU |
| | Target A sequence |
| (SEQ ID NO: 20) | |
| | ACATGGTTGGTGTCACACGT AGG |
| | .G...C.............A |
| | pgRNA A/B |
| (SEQ ID NO: 21) | |
| | AUAUGUUUGGUGUCACACGG |
| | .........T.T...T.... |
| | Target B sequence |
| (SEQ ID NO: 22) | |
| | ATATGTTTGATATCAAACGG GGG |
| | crRNA |
| (SEQ ID NO: 23) | |
| | AUAUGUUUGAUAUCAAACGG |

These results demonstrate that, even subject to the additional constraints, multiple pgRNAs for enAsCas12a could be generated that able to target multiple viral sites simultaneously while maintaining high specificity. These candidates can then be introduced into the computational predictors for on-target enCas12a activity and validated experimentally, where they are expected to strongly suppress reactivation of HIV-1.

TABLE 3
pgRNA spacer candidates for enAsCas12a vs.
HIV-1 proviral genome¹

| | | | Maximum |
|---|---|---|---|
| | | | relative |
| | | Target | predicted |
| | Target | B/C | pgRNA |
| pgRNA | A³ | (gene/ | activity |
| spacer sequence²; | (gene); | feature); | at BLASTn |
| Target A antisense; | Relative | Relative | hits to |
| Target B antisense; | Activity | Activity | human |
| Target C antisense. | (Δ)⁴ | (Δ) | genome⁵ |
| 5′-AGCCUUAUUGAGACUC | 2580 | 513/9598 | (no BLASTn |
| AACCAGU-3′ (SEQ ID | (pol); | 5′-LTR/ | hits with |
| NO: 24); | 0.220 | 3′-LTR); | Tier 1 or |
| 3′-TCGAAATAACTCCGAA | (+0.167) | 0.306 | Tier 2 |
| TTCGTCA-5′ (SEQ ID | | (+0.297) | PAMs) |
| NO: 25); | | | |
| 3′-TCGGGTAAACTCTGAC | | | |
| ATGGTCA-5′ (SEQ ID | | | |
| NO: 26); | | | |
| 3′-TCGGGTAAACTCTGAC | | | |
| ATGGTCA-5′ (SEQ ID | | | |
| NO: 26). | | | |
| 5′-UGAAGAAUCGCAAAAC | 8186 | 6882 | (no BLASTn |
| CAGCCAG-3′ (SEQ ID | (env); | (env); | hits with |
| NO: 27); | 0.439 | 0.238 | Tier 1 or |
| 3′-ACTTCTTAGCGTTTTG | (+0.393) | (+0.118) | Tier 2 |
| GTCGTTC-5′ (SEQ ID | | | PAMs) |
| NO: 28); | | | |
| 3′-AAATCTTAGCGTTTTG | | | |
| GTCGGCC-5′ (SEQ ID | | | |
| NO: 29). | | | |
| 5′-AAAAGCAUCCCCUAGC | 2114 | 5136 | (no BLASTn |
| CUUCCCU-3′ (SEQ ID | (gag); | (vif); | hits with |
| NO: 30); | 0.214 | 0.488 | Tier 1 or |
| 3′-TTCTTTTAAGGGACCG | (+0.186) | (+0.470) | Tier 2 |
| GAAGGGA-5′ (SEQ ID | | | PAMs) |
| NO: 31); | | | |
| 3′-TTTTGGTAGGGGATCG | | | |
| AAAGGGA-5′ (SEQ ID | | | |
| NO: 32). | | | |
| 5′-GUCAUAUUUCCCAUAU | 3731 | 7182 | 0.008526709 |
| UUCCUAU-3′ (SEQ ID | (pol); | (env); | |
| NO: 33); | 0.334 | 0.390 | |
| 3′-TGGTACAAAGGGTACA | (+0.295) | (+0.250) | |
| AAGGAAA-5′ (SEQ ID | | | |
| NO: 34); | | | |
| 3′-GAGTATAAAGGATAAA | | | |
| AAGGATA-5′ (SEQ ID | | | |
| NO: 35); | | | |
| 5′-ACUGACGUAAUACAAC | 3660 | 3441 | 9.76531 x |
| UAACAGA-3′ (SEQ ID | (pol); | (pol); | 10⁻⁶ |
| NO: 36); | 0.216 | 0.205 | |
| 3′-TTACTACATTTTGTTA | (+0.200) | (+0.152) | |
| ATTGTCT-5′ (SEQ ID | | | |
| NO: 37); | | | |
| 3′-TGTCTTCATTATGGTG | | | |
| ATTGTCT-5′ (SEQ ID | | | |
| NO: 38); | | | |

¹pgRNAs have predicted relative activity at both sites >20%; both targets have Tier 1 or Tier 2 PAM sites; and predicted relative activity at BLAST n hits to human genome <1%.
²Underlined at sites where Target A and Target B sequences diverge
³Labelled at the first position of the protospacer 3′- the PAM site, according to Human immunodeficiency virus type 1 (HXB2), complete genome; HIV1/HTLV-III/LAV reference genome
⁴Increase in predicted CRISPR activity by using pgRNA at target A or B/C, compared to using gRNA for target A at target B (or vice versa)
⁵GRCh38.p12 human genome reference sequence

An analysis of 2,372 genomes of RNA viruses in the NCBI Reference Sequence database revealed that these homeologous pairs of Cas13-targetable sites (23 nt) with >70% identity (>16 out of 23 nt) are prevalent across RNA viruses of mammals, birds, and arthropods, and plants: RNA viruses with genomes that are 5,000 nt in length have on average around 30 of such pairs, and those with genomes that are 10,000 nt in length have on average approximately 120, obeying a power law scaling with genome length. For human-hosted RNA viruses, we could identify 19,926 of these homologous target pairs across 89 viruses.

Candidate pgRNA sequences for each pair are then generated in silico by determining what nucleotides at the positions of divergent sequence between the two targets would allow for and maximize predicted activity at both sites (FIG. 1C), which is performed by calculating the expected “mismatch penalties” or reduction of CRISPR RNP activity for those candidates at sites with imperfect complementarity to the spacer sequence. Mismatch penalties have been quantitatively determined for several CRISPR effectors and exhibit a strong dependence on both the type of mismatch (what nucleotides are incorrectly paired) and the position of the mismatch(es) along the target: they have been found to vary not only by type of CRISPR effector but across homologues of the effector derived from different species. For the design of pgRNAs, sequences are selected by computationally maximizing the predicted activity of a single gRNA at multiple viral sites by exploiting well-tolerated mismatch- and position-specific mispairings of the CRISPR effectors to minimize potential reductions of activity at the different sites.

Sequences with predicted biophysical properties that might negatively impact expression or activity such as strong predicted secondary structures or the presence of mononucleotide stretches are then removed from consideration, as are any sequences with more than 65% complementarity with potential “off-targets” in the host genome or transcriptome (with at least 15 nts complementarity for 23 nt Cas13 targets), yielding a final set of pgRNA candidates with high predicted activity at multiple viral sites and effectively no predicted “off-target” activity vs. the host. To illustrate the broad potential applicability of our approach, we found we could design pgRNA candidates for RNA-targeting Cas13d from Ruminococcus flavefaciens XPD3002 (RfxCas13d) with predicted activity at both their targeted sites ranking in the top quartile of all “monovalent” gRNAs for that virus and no significant homology/predicted activity vs. the human transcriptome for 53 of the 59 (+) ssRNA viruses or expressed viral mRNA sequences in the NCBI Reference Sequence database. RfxCas13d, which has been used in CRISPR-based viral diagnostics and was recently demonstrated to disrupt influenza and SARS-CoV-2 virulence in human epithelial cells, was found to exhibit significant tolerance to mismatches relative to other CRISPR effectors and does not require specific flanking sequences next to its targets, so RfxCas13d may represent an optimal effector for antiviral applications in that regard.

To test our hypothesis that pgRNAs targeting to multiple viral sites simultaneously would inhibit viral propagation in vivo during a viral infection better than their monovalent counterparts, we designed pgRNAs for RfxCas13d to target pairs of protospacers found in the tobacco mosaic virus (TMV) and infected Nicotiana benthamiana with a TMV replicon (TRBO-GFP) via Agrobacterium tumefaciens-mediated transformation into its leaves (FIGS. 2A and 2B). The TRBO-GFP replicon, which has previously been used as a model viral infection to validate CRISPR-based antiviral biotechnologies in plants, contains an expression cassette for a modified TMV under the control of a strong constitutive 35S promoter; after transcription, the replication-competent (+) ssRNA virus then can spread cell-to-cell within the leaf as an uncontrolled infectious agent. Here, the TMV coat protein gene in the TRBO-GFP replicon had been replaced with a green fluorescent protein (GFP) gene that allows viral spread to be visually tracked and that we use to as a reporter to quantify overall viral RNA levels in the leaves. At the time of introduction of the TRBO-GFP replicon into the leaves, we also introduce transfer DNAs (T-DNAs) for transient expression of RfxCas13d via A. tumefaciens-mediated transformation and T-DNAs to express either one or two multiplexed gRNAs or pgRNAs (FIG. 2A). The gRNAs and pgRNAs were targeted to the viral replicase gene or movement protein (MP) gene, not the GFP, and designed to avoid the N. bethamiana transcriptome by ensuring they each contain at least 8 mismatches with all sequenced N. bethamiana RNA transcripts (transcriptome assembly v5).

After three days, plants expressing one of six different monovalent gRNAs showed viral RNA levels in their leaves reduced to approximately 10% to 25% of those in plants that were not targeting TMV via Cas13 (FIG. 2C-E). Plants expressing a single monovalent gRNAs exhibited less viral suppression than those expressing a single pgRNA, who were able to robustly supress viral spread (FIGS. 2C and 2D) and viral gene expression (3.4%±0.4% (95% confidence) GFP mRNA levels relative to plants expressing gRNA-NT). This performance by the pgRNA is remarkable considering that the pgRNA spacer sequence contains three imperfectly (noncanonically) complementary or mis-paired nucleotides with each of its two targets, so its ability to reduce viral RNA more than perfectly matched gRNAs for each of its target suggests that its “polyvalency” or ability to recognize multiple targets on the virus can compensate for potential reductions in activity or “mismatch penalties” at those targets in vivo. In fact, the plants expressing the pgRNA exhibited reduced viral levels similar to those plants undergoing multiplexed expression of two of their “monovalent” counterparts (2% to 8% viral RNA), while multiplexed expression of two sets of three pgRNAs (together targeting four viral targets simultaneously with two guides) further reduced viral RNA levels by an order of magnitude, to 0.3%-0.5% viral RNA in the leaves compared to untreated plants. A third multiplexed pgRNA set “only” reducing viral RNA levels to 5%, levels equivalent to multiplexed monovalent gRNAs, although this may be a result of a predicted partial base-pairing interaction between the two multiplexed pgRNAs in that set, that is known to affect CRISPR activity. We found that the antiviral effect of pgRNAs is mediated by the targeted RNAse activity of Cas13d (FIG. 2D), although treatments with a catalytically inactive Cas13d variant (dCas13d) exhibited modest (10-40%) reduction of viral RNA levels in N. bethamiana through some as-yet-unknown mechanism. We otherwise found no evidence of disruption of “off-target” cellular RNA levels. The significant inhibition of viral propagation and spread during infection by pgRNAs therefore suggests that polyvalent targeting of viruses using pgRNAs might represent a superior paradigm for gRNA design in CRISPR antiviral applications and further highlights the potential for CRISPR effectors as viral prophylactic and treatments in plants and other organisms.

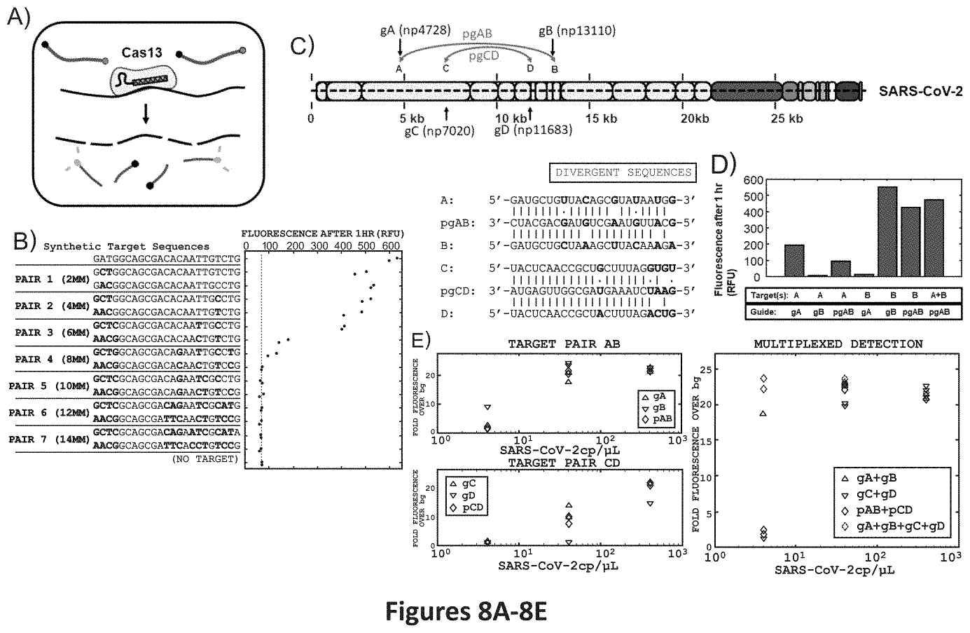

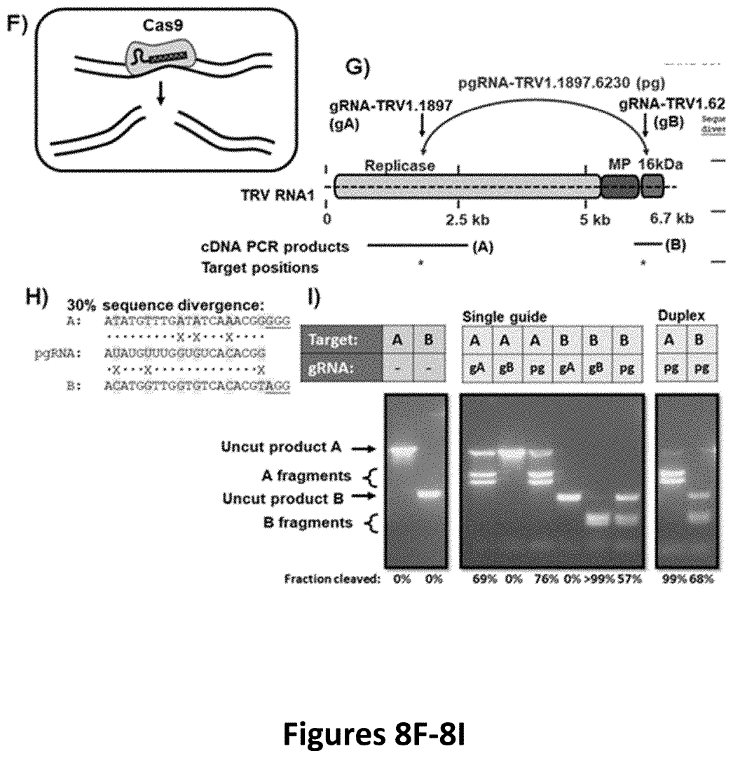

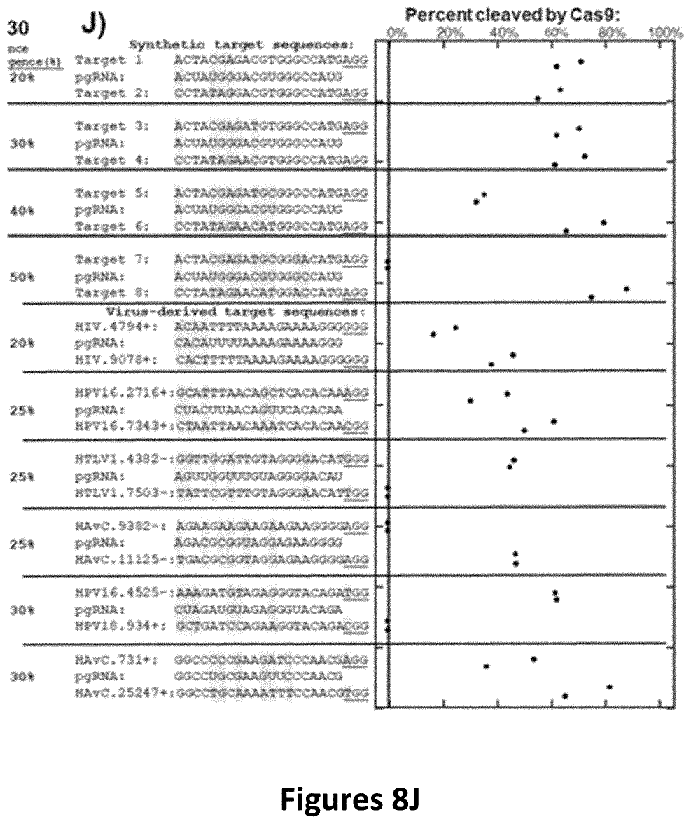

After target recognition and cleavage, many Cas13 variants undergo a conformational change and exhibit “collateral activity” or a non-specific RNAse activity that has been used for applications in viral diagnostics such as SHERLOCK (FIG. 8A), including in a diagnostic assay for SARS-CoV-2 (FIG. 8C), the (+) ssRNA coronavirus responsible for the COVID19 respiratory infection. In viral detection systems using CRISPR effectors like SHERLOCK, it has been found that multiplexed use of multiple gRNAs improves viral detection sensitivity and so we sought to determine whether pgRNAs could be used for these in vitro applications to trigger collateral activity at multiple viral targets, simultaneously, with fewer components. SHERLOCK and the activation of collateral activity has been reported to be sensitive to single-nucleotide polymorphisms in their targets, however we found we could engineer single pgRNAs that could successfully trigger Cas13 collateral activity at multiple synthetic and SARS-CoV-2 derived RNA targets that diverged by up to 25% (6 out of 23 nt), and which could even exhibit collateral activity at targets with up to 4 nt mismatches with the gRNA spacers (FIG. 8B). This polyvalently-triggered collateral activity was specific to the engineered pgRNAs: regular (perfectly matched) “monovalent” gRNAs exhibited no cross-reactivity in vitro at paired sites with such high sequence divergence (FIG. 8D).

To assess whether pgRNAs might be suitable for in vitro viral diagnostics, We generated a series of 23 pgRNAs with high predicted activity at 15 target pairs found in SARS-CoV-2, then screened their collateral activity in the presence of their SARS-CoV-2 RNA targets and compared those results with the combined activity their perfectly matched monovalent gRNA counterparts (30 separate gRNAs). We found that each of the pgRNAs tested exhibited collateral activity at levels similar to or higher than their combined monovlanent gRNA counterparts with both targets present in the same sample, and no off-site collateral activity was detected in the presence of non-targeted RNA sequences, universal human reference RNA (10 human cell lines; ThermoFisher Scientific), or human lung total RNA (ThermoFisher Scientific) (3 μg RNA). We then assessed their limits of detection (LoD) in a SHERLOCK-type assay using Cas13 and the best-performing pgRNAs, and found that Cas13 with single pgRNAs (recognizing two sites) or two pgRNAs (recognizing four) could robustly generate detectable signals in samples initially containing 40 cp/uL heat-inactivated SARS-CoV-2 (clinically relevant LoD for SARS-CoV-2 is often considered to be 1000 cp/uL) (FIG. 8E), performing as well as their monovalent counterparts and even some multiplexed monovalent gRNAs in this assay, with fewer components, suggesting the suitability of pgRNAs for in vitro multiplexed viral detection applications of multiple viral targets.

Last, we sought to determine whether the design principles we use for pgRNAs could be applied to gRNAs of other types of CRISPR effectors like the Cas9 effector from Streptococcus pyogenes (SpyCas9), which recognizes and introduces double-strand breaks into dsDNA targets (FIG. 8F). We designed pgRNAs to target homeologous pairs of synthetic or virally derived DNA protospacers with sequence divergence up to 50%, that is, differing at up to 10 of the 20 bp sites in the SpyCas9 protospacers and measured the cleavage activity of the Cas9 RNPs at those sites ex vivo (FIGS. 8G-8J). As with Cas13, while regular guide RNAs exhibited no cross-reactivity at paired sites with such high sequence divergence, SpyCas9 RNPs with pgRNAs could consistently cleave both targets even when paired sequences diverged by up to 40% (FIGS. 8A-J). In cases where the pgRNA only exhibited activity at one target, those targets could still possess up to 5 mismatches between the pgRNA spacer and the protospacer. Additionally, we found that including a leading 5′-rG on the spacer, a condition thought to result in greater specificity in CRISPR activity for gene editing applications, consequently reduced pgRNA activity at both sites, which further highlights the idea that conditions optimized for precision gene editing might not be ideal for maximizing CRISPR activity during antiviral applications. Hence, by optimizing the tolerance for mismatches between the spacer sequence and targeted sites, we show that pgRNAs can also be engineered to promote high levels of SpyCas9 cleavage activity at multiple targeted DNA sequences simultaneously ex vivo. SpyCas9 has been for cellular treatments of retroviruses and recently used to treat an animal model of herpesvirus infection, and the results here demonstrate the promise and potential utility of pgRNAs for the treatment of DNA viruses as well.

Referring now to FIG. 8A, the image depicts a representation of Cas binding and activity. After recognizing a target, Cas13 exhibits nonspecific RNAse activity; nonspecific degradation of a fluorescent reporter RNA results in a fluorescent signal that can be detected in viral diagnostic assays.

Referring now to FIG. 8B, the image displays a table representing detectable collateral activity is stimulated by Cas13 in vitro at targets with sequence divergence up to 25%.

Referring now to FIG. 8C, the image depicts example pgRNAs designed to target (+) ssRNA virus SARS-CoV-2.

Referring now to FIG. 8D, the image depicts a graph displaying data indicating monovalent gRNAs exhibit no cross-reactive collateral activity, while pgRNAs exhibit collateral activity in the presence of either SARS-CoV-2 target.

Referring now to FIG. 8E, the image depicts graphs displaying data from a SHERLOCK-type Cas13 viral diagnostic assay, Cas13 with single pgRNAs (recognizing two sites, left) or two pgRNAs (recognizing four, right) could robustly generate detectable signals in the presence of samples initially containing 40 cp/uL heat-inactivated SARS-CoV-2 (clinically relevant LoD for SARS-CoV-2 is often considered to be 1000 cp/uL).

Referring now to FIG. 8F, the image depicts a representation showing Cas9 recognizes and cleaves dsDNA.

Referring now to FIG. 8G, the image depicts a pgRNA (pg) and its two “monovalent” counterpart gRNAs (gA and gB) for Cas9 from S. pyogenes that was designed to target two sequences derived from the Tobacco Rattle Virus (TRV) segment 1 (RNA1) at positions 1897 (target A) and 6230 (target B).

Referring now to FIG. 8H, the image depicts a sequence comparison showing divergence of targets A and B, which differ by 6 of the 20 nt (30%) in their protospacer region, and 1 out of 3 within their protospacer adjacent motif (PAM) region (underlined).

Referring now to FIG. 8I, the gel assay demonstrates that monovalent guides exhibit no cross-reactivity at homologous sites, while the pgRNA exhibits robust cleavage activity at both sites. pgRNA activity is enhanced with a crRNA:tracrRNA duplex compared to a chimeric “single guide” RNA.

Referring now to FIG. 8J, the image depicts pgRNAs that may be generated for SpyCas9 to exhibit robust cleavage activity ex vivo at pairs of synthetic (upper) and virally derived (lower) targets with sequences diverging by up to 40%, suggestive of their potential for activity against dsDNA viruses. HIV: Human Immunodeficiency Virus type 1; HPV16: Human papillomavirus type 16; HPV18: Human papillomavirus type 18; HTLV1: Human T-lymphotropic virus 1; HAvC: Human Adenovirus C.

The CRISPR effector proteins used in biotechnological applications were originally found in bacteria and archaea as an antiviral mechanism to degrade foreign DNA and RNA, and so some tolerance to sequence variation in their targets is likely beneficial for this purpose. In gene editing applications, this tolerance is suppressed to the greatest extent possible using a number of strategies to prevent degradation and mutations at any sequence not exactly matching the gRNA spacer sequence. Rather, in a new gRNA design paradigm for antiviral applications, we show that the polyvalent targeting of viruses by single engineered gRNAs—optimized based on the CRISPR effector's natural position- and sequence-determined tolerance for mismatches for activity at the homologous target pairs that are abundant in viral genomes—can drive robust CRISPR activity at specific targeted pairs simultaneously in vitro/ex vivo, can exhibit stronger viral suppression during infection of a higher organism relative to “monovalent” targeting, and may in fact be optimal for applications of CRISPR antiviral diagnostics, prophylactics, and therapeutics.

1. A method to determine a pgRNA sequence comprising:
identifying two or more target sequences in a viral genome for recognition by a Cas effector;
for each target sequence of the two or more target sequences, calculating a homology score comprising aligning said target sequence with each other target sequence of the two or more target sequences;
determining one or more target pairs based at least in part on the homology score, wherein each target pair comprises a first target sequence and a second target sequence of the two or more target sequences having the homology score calculated as greater than or equal to 60% sequence identity;
generating a pgRNA template for at least one of the one or more target pairs, wherein the pgRNA template has a complementary sequence to the first target sequence, the second target sequences, or a convergent sequence.
generating a relative activity score for each of one or more pgRNA templates by comparing the pgRNA template to a complementary sequence to the first target sequence and a complementary sequence to a second nucleotide sequence present in a different viral genome, a mutant viral genome, or both, wherein each pgRNA template comprises a sequence of nucleotides;
determining whether to calculate an off-target score for each pgRNA template based at least in part on the relative activity score generated for said pgRNA template; and
determining the pgRNA sequence based at least in part on the relative activity score for each pgRNA template, the off-target score, or both.

2. The method of claim 1, wherein the two or more target sequences are RNA, DNA or both.

3. The method of claim 2, wherein the two or more target sequences are RNA.

4. The method of claim 1, wherein identifying the two or more target sequences in the viral genome comprises:
determining a sequence position for each of one or more protospacer motifs present in the viral genome based at least in part on the CAS effector, wherein each of the one or more protospacer motifs comprise an adjacent sequence of nucleotides; and
assigning at least one sequence position as a protospacer position; and
identifying the two or more target sequences as a sequence of nucleotides immediately downstream of the protospacer position.

5. The method of claim 1, wherein the Cas effector is enAsCas12a.

6. The method of claim 4, wherein the one or more protospacer motifs are from the group consisting of: TTYN, CTTV, RTTC, TATM, CTCC, TCCC, TACA, RTTS, TATA, TGTV, ANCC, CVCC, TGCC, GTCC, TTAC, or combinations thereof.

7. The method of claim 6, wherein the one or more protospacer motifs are from the group consisting of: TTYN, CTTV, RTTC, TATM, CTCC, TCCC, TACA, or combinations thereof.

8. The method of claim 1, wherein the different viral genome and the viral genome are included in a viral family.

9. The method of claim 8, wherein the viral family is coronaviruses.

10. The method of claim 1, wherein comparing the pgRNA template to the complementary sequence to the first nucleotide sequence and the complementary sequence to the second nucleotide sequence present in the different viral genome, the mutant viral genome, or both:
determining a first sequence identify for the pgRNA template to the complementary sequence to the first nucleotide sequence and a second sequence identity for the pgRNA template to the complementary sequence to the second nucleotide sequence, wherein the first sequence identity and the second sequence identity are calculated based on a BLAST alignment, and wherein the relative activity score is based at least in part on the first sequence identity and the second sequence identity.

11. The method of claim 10, wherein calculating the off-target score is performed only for the pgRNA templates having calculated the first sequence identity as greater than about 60% and the second sequence identity as greater than about 60%.

12. The method of claim 11, wherein calculating the off-target score is performed only for the pgRNA templates having calculated the first sequence identity as greater than about 90% and the second sequence identity as greater than about 90%.

13. The method of claim 11, wherein calculating the off-target score is based at least in part on comparing each of the one or more pgRNA templates to a human genome sequence or a human transcriptome sequence.

14. The method of claim 1, wherein determining the pgRNA sequence is based at least in part on a region of interest comprising a sequence of adjacent nucleotides present in the viral genome.

15. The method of claim 1, wherein, each target pair comprises a first target sequence and a second target sequence of the two or more target sequences having the homology score calculated as greater than or equal to 75% sequence identity.

16. A pgRNA having a pgRNA sequence determined according to the method of claim 1.

17. The pgRNA of claim 16, wherein the pgRNA sequence is determined based on identifying two or more target sequences in a coronavirus genome.

18. The pgRNA of claim 17, wherein the coronavirus genome is SARS-CoV-2.

19. The pgRNA of claim 16, wherein the pgRNA sequence comprises UAACCAUUGUUCGCUGUAACAGUAUCA (SEQ ID NO: 4).

20. A method for treating a viral infection in a patient comprising, -delivering to a patient in need thereof a composition comprising the pgRNA of claim 16.

21. The method of claim 20, wherein the patient displays symptoms of Covid-19.


SEQUENCE LISTING (95 sequences)

SEQ ID NO: 1 - DNA, 23 bp, Human mastadenovirus C
ggcctgcaaaatttccaacgtgg

SEQ ID NO: 2 - RNA, 29 nt, Artificial Sequence
  Description of Artificial Sequence Synthetic oligonucleotide
accccuaccaacuggucgggguuugaaac

SEQ ID NO: 3 - RNA, 20 nt, Artificial Sequence
  Description of Artificial Sequence Synthetic oligonucleotide
uaauuucuacucuuguagau

SEQ ID NO: 4 - RNA, 27 nt, Artificial Sequence
  Description of Artificial Sequence Synthetic oligonucleotide
uaaccauuguucgcuguaacaguauca

SEQ ID NO: 5 - RNA, 27 nt, Severe acute respiratory syndrome coronavirus 2
ugaugcuguuacagcguauaaugguua

SEQ ID NO: 6 - RNA, 27 nt, Severe acute respiratory syndrome coronavirus 2
ugauaguguuacagugaagaaugguuc

SEQ ID NO: 7 - RNA, 27 nt, Artificial Sequence
  Description of Artificial Sequence Synthetic oligonucleotide
agauaaacguucuaugcuuuaacagca

SEQ ID NO: 8 - RNA, 27 nt, Severe acute respiratory syndrome coronavirus 2
ugcuguuacagcguauaaugguuaucu

SEQ ID NO: 9 - RNA, 27 nt, Severe acute respiratory syndrome coronavirus 2
ugcugcuaaagcuuacaaagauuaucu

SEQ ID NO: 10 - RNA, 27 nt, Artificial Sequence
  Description of Artificial Sequence Synthetic oligonucleotide
acauuguuggcaaguucagcuacugua

SEQ ID NO: 11 - RNA, 27 nt, Severe acute respiratory syndrome coronavirus 2
ugcagaagcugaacuugcaaagaaugu

SEQ ID NO: 12 - RNA, 27 nt, Severe acute respiratory syndrome coronavirus 2
uucaguagcugcacuuacuaacaaugu

SEQ ID NO: 13 - RNA, 27 nt, Artificial Sequence
  Description of Artificial Sequence Synthetic oligonucleotide
auauaguaguagauuaccagaagcauc

SEQ ID NO: 14 - RNA, 27 nt, Severe acute respiratory syndrome coronavirus 2
gaugcuucugguaagccaguaccauau

SEQ ID NO: 15 - RNA, 27 nt, Severe acute respiratory syndrome coronavirus 2
gcugcuucugguaaucuauuacuagau

SEQ ID NO: 16 - RNA, 27 nt, Artificial Sequence
  Description of Artificial Sequence Synthetic oligonucleotide
uaaauugcaaccugucauaaacguguc

SEQ ID NO: 17 - RNA, 27 nt, Severe acute respiratory syndrome coronavirus 2
gagaccuuuaugacaaguugcaauuua

SEQ ID NO: 18 - RNA, 27 nt, Severe acute respiratory syndrome coronavirus 2
gcuacguguauaacacguugcaauuua

SEQ ID NO: 19 - RNA, 20 nt, Artificial Sequence
  Description of Artificial Sequence Synthetic oligonucleotide
acaugguuggugucacacgu

SEQ ID NO: 20 - DNA, 23 bp, Tobacco rattle virus
acatggttggtgtcacacgtagg

SEQ ID NO: 21 - RNA, 20 nt, Artificial Sequence
  Description of Artificial Sequence Synthetic oligonucleotide
auauguuuggugucacacgg

SEQ ID NO: 22 - DNA, 23 bp, Tobacco rattle virus
atatgtttgatatcaaacggggg

SEQ ID NO: 23 - RNA, 20 nt, Artificial Sequence
  Description of Artificial Sequence Synthetic oligonucleotide
auauguuugauaucaaacgg

SEQ ID NO: 24 - RNA, 23 nt, Artificial Sequence
  Description of Artificial Sequence Synthetic oligonucleotide
agccuuauugagacucaaccagu

SEQ ID NO: 25 - DNA, 23 bp, Human immunodeficiency virus 1
actgcttaagcctcaataaagct

SEQ ID NO: 26 - DNA, 23 bp, Human immunodeficiency virus 1
actggtacagtctcaaatgggct

SEQ ID NO: 27 - RNA, 23 nt, Artificial Sequence
  Description of Artificial Sequence Synthetic oligonucleotide
ugaagaaucgcaaaaccagccag

SEQ ID NO: 28 - DNA, 23 bp, Human immunodeficiency virus 1
cttgctggttttgcgattcttca

SEQ ID NO: 29 - DNA, 23 bp, Human immunodeficiency virus 1
ccggctggttttgcgattctaaa

SEQ ID NO: 30 - RNA, 23 nt, Artificial Sequence
  Description of Artificial Sequence Synthetic oligonucleotide
aaaagcauccccuagccuucccu

SEQ ID NO: 31 - DNA, 23 bp, Human immunodeficiency virus 1
agggaaggccagggaattttctt

SEQ ID NO: 32 - DNA, 23 bp, Human immunodeficiency virus 1
agggaaagctaggggatggtttt

SEQ ID NO: 33 - RNA, 23 nt, Artificial Sequence
  Description of Artificial Sequence Synthetic oligonucleotide
gucauauuucccauauuuccuau

SEQ ID NO: 34 - DNA, 23 bp, Human immunodeficiency virus 1
aaaggaaacatgggaaacatggt

SEQ ID NO: 35 - DNA, 23 bp, Human immunodeficiency virus 1
ataggaaaaataggaaatatgag

SEQ ID NO: 36 - RNA, 23 nt, Artificial Sequence
  Description of Artificial Sequence Synthetic oligonucleotide
acugacguaauacaacuaacaga

SEQ ID NO: 37 - DNA, 23 bp, Human immunodeficiency virus 1
tctgttaattgttttacatcatt

SEQ ID NO: 38 - DNA, 23 bp, Human immunodeficiency virus 1
tctgttagtggtattacttctgt

SEQ ID NO: 39 - RNA, 23 nt, Severe acute respiratory syndrome coronavirus 2
ugaugcuguuacagcguauaaug

SEQ ID NO: 40 - RNA, 23 nt, Severe acute respiratory syndrome coronavirus 2
agaugcugcuaaagcuuacaaag

SEQ ID NO: 41 - RNA, 23 nt, Tobacco mosaic virus
ugagcaguuuuauacugcaaugg

SEQ ID NO: 42 - RNA, 23 nt, Artificial Sequence
  Description of Artificial Sequence Synthetic oligonucleotide
ccauugccguaugaaacugcucu

SEQ ID NO: 43 - RNA, 23 nt, Tobacco mosaic virus
agagcaguuucauauggcgacgg

SEQ ID NO: 44 - RNA, 23 nt, Tobacco mosaic virus
gaggaggugugagcgugugucug

SEQ ID NO: 45 - RNA, 23 nt, Artificial Sequence
  Description of Artificial Sequence Synthetic oligonucleotide
cagagacccgcugacaucucccc

SEQ ID NO: 46 - RNA, 23 nt, Tobacco mosaic virus
gugaagaugucagcggguuucug

SEQ ID NO: 47 - RNA, 23 nt, Tobacco mosaic virus
acggaguuccgggcuguggauaa

SEQ ID NO: 48 - DNA, 23 bp, Human mastadenovirus C
ggcccccgaagatcccaacgagg

SEQ ID NO: 49 - RNA, 20 nt, Artificial Sequence
  Description of Artificial Sequence Synthetic oligonucleotide
ggccugcgaaguucccaacg

SEQ ID NO: 50 - RNA, 23 nt, Artificial Sequence
  Description of Artificial Sequence Synthetic oligonucleotide
uuaugcacagccgggaacuccgu

SEQ ID NO: 51 - DNA, 23 bp, Artificial Sequence
  Description of Artificial Sequence Synthetic oligonucleotide
gatggcagcgacacaattgtctg

SEQ ID NO: 52 - DNA, 23 bp, Artificial Sequence
  Description of Artificial Sequence Synthetic oligonucleotide
gctggcagcgacacaattgtctg

SEQ ID NO: 53 - DNA, 23 bp, Artificial Sequence
  Description of Artificial Sequence Synthetic oligonucleotide
gacggcagcgacacaattgtctg

SEQ ID NO: 54 - DNA, 23 bp, Artificial Sequence
  Description of Artificial Sequence Synthetic oligonucleotide
gctggcagcgacacaattgcctg

SEQ ID NO: 55 - DNA, 23 bp, Artificial Sequence
  Description of Artificial Sequence Synthetic oligonucleotide
aacggcagcgacacaattgtctg

SEQ ID NO: 56 - DNA, 23 bp, Artificial Sequence
  Description of Artificial Sequence Synthetic oligonucleotide
gctcgcagcgacacaattgcctg

SEQ ID NO: 57 - DNA, 23 bp, Artificial Sequence
  Description of Artificial Sequence Synthetic oligonucleotide
aacggcagcgacacaactgtctg

SEQ ID NO: 58 - DNA, 23 bp, Artificial Sequence
  Description of Artificial Sequence Synthetic oligonucleotide
gctcgcagcgacagaattgcctg

SEQ ID NO: 59 - DNA, 23 bp, Artificial Sequence
  Description of Artificial Sequence Synthetic oligonucleotide
aacggcagcgacacaactgtccg

SEQ ID NO: 60 - DNA, 23 bp, Artificial Sequence
  Description of Artificial Sequence Synthetic oligonucleotide
gctcgcagcgacagaatcgcctg

SEQ ID NO: 61 - DNA, 23 bp, Artificial Sequence
  Description of Artificial Sequence Synthetic oligonucleotide
aacggcagcgatacaactgtccg

SEQ ID NO: 62 - DNA, 23 bp, Artificial Sequence
  Description of Artificial Sequence Synthetic oligonucleotide
gctcgcagcgacagaatcgcatg

SEQ ID NO: 63 - DNA, 23 bp, Artificial Sequence
  Description of Artificial Sequence Synthetic oligonucleotide
aacggcagcgattcaactgtccg

SEQ ID NO: 64 - DNA, 23 bp, Artificial Sequence
  Description of Artificial Sequence Synthetic oligonucleotide
gctcgcagcgacagaatcgcata

SEQ ID NO: 65 - DNA, 23 bp, Artificial Sequence
  Description of Artificial Sequence Synthetic oligonucleotide
aacggcagcgattcacctgtccg

SEQ ID NO: 66 - RNA, 23 nt, Severe acute respiratory syndrome coronavirus 2
gaugcuguuacagcguauaaugg

SEQ ID NO: 67 - RNA, 23 nt, Artificial Sequence
  Description of Artificial Sequence Synthetic oligonucleotide
gcauuguaagcuguagcagcauc

SEQ ID NO: 68 - RNA, 23 nt, Severe acute respiratory syndrome coronavirus 2
gaugcugcuaaagcuuacaaaga

SEQ ID NO: 69 - RNA, 23 nt, Severe acute respiratory syndrome coronavirus 2
uacucaaccgcugcuuuaggugu

SEQ ID NO: 70 - RNA, 23 nt, Artificial Sequence
  Description of Artificial Sequence Synthetic oligonucleotide
gaaucuaaaguagcgguugagua

SEQ ID NO: 71 - RNA, 23 nt, Severe acute respiratory syndrome coronavirus 2
uacucaaccgcuacuuuagacug

SEQ ID NO: 72 - DNA, 23 bp, Artificial Sequence
  Description of Artificial Sequence Synthetic oligonucleotide
actacgagacgtgggccatgagg

SEQ ID NO: 73 - RNA, 20 nt, Artificial Sequence
  Description of Artificial Sequence Synthetic oligonucleotide
acuaugggacgugggccaug

SEQ ID NO: 74 - DNA, 23 bp, Artificial Sequence
  Description of Artificial Sequence Synthetic oligonucleotide
cctataggacgtgggccatgagg

SEQ ID NO: 75 - DNA, 23 bp, Artificial Sequence
  Description of Artificial Sequence Synthetic oligonucleotide
actacgagatgtgggccatgagg

SEQ ID NO: 76 - DNA, 23 bp, Artificial Sequence
  Description of Artificial Sequence Synthetic oligonucleotide
cctatagaacgtgggccatgagg

SEQ ID NO: 77 - DNA, 23 bp, Artificial Sequence
  Description of Artificial Sequence Synthetic oligonucleotide
actacgagatgcgggccatgagg

SEQ ID NO: 78 - DNA, 23 bp, Artificial Sequence
  Description of Artificial Sequence Synthetic oligonucleotide
cctatagaacatgggccatgagg

SEQ ID NO: 79 - DNA, 23 bp, Artificial Sequence
  Description of Artificial Sequence Synthetic oligonucleotide
actacgagatgcgggacatgagg

SEQ ID NO: 80 - DNA, 23 bp, Artificial Sequence
  Description of Artificial Sequence Synthetic oligonucleotide
cctatagaacatggaccatgagg

SEQ ID NO: 81 - DNA, 23 bp, Human immunodeficiency virus 1
acaattttaaaagaaaagggggg

SEQ ID NO: 82 - RNA, 20 nt, Artificial Sequence
  Description of Artificial Sequence Synthetic oligonucleotide
cacauuuuaaaagaaaaggg

SEQ ID NO: 83 - DNA, 23 bp, Human immunodeficiency virus 1
cactttttaaaagaaaagggggg

SEQ ID NO: 84 - DNA, 23 bp, Alphapapillomavirus 9
gcatttaacagctcacacaaagg

SEQ ID NO: 85 - RNA, 20 nt, Artificial Sequence
  Description of Artificial Sequence Synthetic oligonucleotide
cuacuuaacaguucacacaa

SEQ ID NO: 86 - DNA, 23 bp, Alphapapillomavirus 9
ctaattaacaaatcacacaacgg

SEQ ID NO: 87 - DNA, 23 bp, Primate T-lymphotropic virus 1
ggttggattgtaggggacatggg

SEQ ID NO: 88 - RNA, 20 nt, Artificial Sequence
  Description of Artificial Sequence Synthetic oligonucleotide
aguugguuuguaggggacau

SEQ ID NO: 89 - DNA, 23 bp, Primate T-lymphotropic virus 1
tattcgtttgtagggaacattgg

SEQ ID NO: 90 - DNA, 23 bp, Human mastadenovirus C
agaagaagaagaagaaggggagg

SEQ ID NO: 91 - RNA, 20 nt, Artificial Sequence
  Description of Artificial Sequence Synthetic oligonucleotide
agacgcgguaggagaagggg

SEQ ID NO: 92 - DNA, 23 bp, Human mastadenovirus C
tgacgcggtaggagaaggggagg

SEQ ID NO: 93 - DNA, 23 bp, Alphapapillomavirus 9
aaagatgtagagggtacagatgg

SEQ ID NO: 94 - RNA, 20 nt, Artificial Sequence
  Description of Artificial Sequence Synthetic oligonucleotide
cuagauguagaggguacaga

SEQ ID NO: 95 - DNA, 23 bp, Alphapapillomavirus 7
gctgatccagaaggtacagacgg